(19) United States
(12) Patent Application Publication
(10) Pub. No.: US 2021/0032639 A1
(43) Pub. Date: Feb. 4, 2021

(54) MODIFIED 5'-UNTRANSLATED REGION (UTR) SEQUENCES FOR INCREASED PROTEIN PRODUCTION IN BACILLUS

(71) Applicant: DANISCO US INC, Palo Alto, CA (US)

(72) Inventors: Dennis DE LANGE, Leiden (NL); Ryan L. FRISCH, Newark, DE (US); Helen Olivia MASSON, La Jolla, CA (US)

(73) Assignee: Danisco US Inc., Palo Alto, CA (US)

(21) Appl. No.: 16/647,191

(22) PCT Filed: Sep. 5, 2018
(86) PCT No.: PCT/US18/49470

§ 371 Date: Mar. 13, 2020

Related U.S. Application Data
(60) Provisional application No. 62/558,304, filed on Sep. 13, 2017.

(51) Int. Cl. C12N 15/75; C12N 9/28

(52) U.S. Cl. CPC C12N 15/75; C12N 2840/55; C12N 9/2417; C12Y 302/01001

(57) ABSTRACT

The present disclosure is generally modified Bacillus strains and host cells thereof capable of producing increased amounts of industrially relevant proteins of interest. Other embodiments of the disclosure are related to isolated polynucleotides comprising modified Bacillus subtilis aprE 5′-untranslated region (5′-UTR) nucleic acid sequences, vectors thereof, DNA (expression) constructs thereof, modified Bacillus (daughter) cells thereof, and methods of making and using the same.

### CROSS REFERENCE TO RELATED APPLICATION

This application claims the benefit of U.S. Provisional Patent No. 62/558,304, filed Sep. 13, 2017, which is hereby incorporated by reference in its entirety.

### FIELD

The present disclosure is generally related to the fields of bacteriology, microbiology, genetics, molecular biology, enzymology, industrial protein production and the like. More particularly, certain embodiments of the disclosure are related to modified Bacillus strains and host cells thereof capable of producing increased amounts of industrially relevant proteins of interest. Other embodiments of the disclosure are related to isolated polynucleotides comprising modified Bacillus subtilis aprE 5′-untranslated region (5′-UTR) nucleic acid sequences, vectors thereof, DNA (expression) constructs thereof, modified Bacillus (daughter) cells thereof, and methods of making and using the same.

### REFERENCE TO A SEQUENCE LISTING

The contents of the electronic submission of the text file Sequence Listing, named “20180823_NB41250WOPCT_SequenceListing_ST25.txt” was created on Aug. 23, 2018 and is 38 KB in size, which is hereby incorporated by reference in its entirety.

### BACKGROUND

Gram-positive bacteria such as Bacillus subtilis, Bacillus licheniformis, Bacillus amyloliquefaciens and the like are frequently used as microbial factories for the production of industrial relevant proteins, due to their excellent fermentation properties and high yields (e.g., up to 25 grams per liter culture; Van Dijl and Hecker, 2013). For example, B. subtilis is well known for its production of α-amylases (Jensen et al., 2000; Raul et al., 2014) and proteases (Brode et al., 1996) necessary for food, textile, laundry, medical instrument cleaning, pharmaceutical industries and the like (Westers et al., 2004). Because these non-pathogenic Gram-positive bacteria produce proteins that completely lack toxic by-products (e.g., lipopolysaccharides; LPS, also known as endotoxins) they have obtained the “Qualified Presumption of Safety” (QPS) status of the European Food Safety Authority, and many of their products gained a “Generally Recognized As Safe” (GRAS) status from the US Food and Drug Administration (Olempska-Beer et al., 2006; Earl et al., 2008; Caspers et al., 2010).

Thus, the production of proteins (e.g., enzymes, antibodies, receptors, etc.) in microbial host cells is of particular interest in the biotechnological arts. Likewise, the optimization of Bacillus host cells for the production and secretion of one or more protein(s) of interest is of high relevance, particularly in the industrial biotechnology setting, wherein small improvements in protein yield are quite significant when the protein is produced in large industrial quantities. More particularly, B. licheniformis is a Bacillus species host cell of high industrial importance, and as such, the ability to genetically modify and engineer B. licheniformis host cells for enhanced/increased protein expression/production is highly desirable for construction of new and improved B. licheniformis production strains.

Thus, the disclosure set forth herein is related to the highly desirable and unmet needs of obtaining and constructing Bacillus host cells (e.g., protein production host cells, cell factories) having increased protein production capabilities, and the like.

### SUMMARY

The instant disclosure is generally related to modified Bacillus strains and host cells thereof capable of producing increased amounts of industrially relevant proteins of interest. More particularly, certain embodiments of the disclosure are directed to an isolated polynucleotide comprising a modified Bacillus subtilis aprE 5′-untranslated region (mod-5′-UTR) nucleic acid sequence derived from a wild-type Bacillus subtilis aprE 5′-untranslated region (WT-5′-UTR) nucleic acid sequence SEQ ID NO: 1. In certain embodiments, the mod-5′-UTR comprises SEQ ID NO: 2. In other embodiments, the mod-5′-UTR further comprises an upstream (5′) promoter region nucleic acid sequence 5′ and operably linked to the mod-5′-UTR.

In another embodiment, the mod-5′-UTR further comprises a downstream (3′) open reading frame (ORF) nucleic acid sequence encoding a protein of interest, wherein the ORF sequence is 3′ and operably linked to the mod-5′-UTR. In certain other embodiments, the isolated polynucleotide comprises Formula (I) in the 5′ to 3′ direction:

[Pro][mod-5′-UTR][ORF]; (I):

wherein [Pro] is a promoter region nucleic acid sequence operable in a Bacillus sp. cell, [mod-5′-UTR] is a modified B. subtilis aprE 5′ untranslated region (mod-5′-UTR) nucleic acid sequence and [ORF] is an open reading frame nucleic acid sequence encoding a protein of interest (POI), wherein the [Pro], [mod-5′-UTR] and [ORF] nucleic acid sequences are operably linked. In other embodiments, a vector or DNA expression construct comprises an isolated polynucleotide of the disclosure. In yet other embodiments, a recombinant Bacillus sp. cell comprises an isolated polynucleotide of the disclosure. In certain embodiments, the Bacillus sp. is a Bacillus licheniformis cell.

In another embodiment, the disclosure is related to an isolated polynucleotide comprising a modified Bacillus sp. 5′-UTR (mod-5′-UTR) nucleic acid sequence derived from a wild-type Bacillus sp. 5′-UTR (WT-5′-UTR) sequence, the isolated modified polynucleotide comprising in the 5′ to 3′ direction the nucleic acid sequences of Formula (II) in operable combination:

[TIS][mod-5′ UTR][tss codon] (II):

wherein [TIS] is transcription initiation site (TIS), [mod-5′-UTR] comprises a modified B. subtilis 5′-UTR nucleic acid sequence and [tss codon] is a three (3) nucleotide translation start site (tss) codon. In certain embodiments, the [mod-5′-UTR] sequence comprises SEQ ID NO: 2. In another embodiment, the polynucleotide further comprises (a) a nucleic acid promoter sequence upstream (5′) and operably linked to the [TIS], which promoter sequence is operable in a Bacillus sp. cell and (b) an ORF nucleic acid sequence downstream (3′) and operably linked to the tss codon, wherein the ORF sequence encodes a POI. In other embodiments, a vector or DNA expression construct comprises an isolated polynucleotide of the disclosure. In particular embodiments, a recombinant Bacillus sp. cell comprises the polynucleotide of Formula (II). In certain other embodiments, the Bacillus sp. cell is a Bacillus licheniformis cell.

In another embodiment, the disclosure is directed to an isolated polynucleotide comprising nucleic acid sequences of Formula (III) in the 5′ to 3′ direction and in operable combination,

[5′-HR][TIS][mod-5′-UTR][tss codon][3′-HR], (III):

wherein [TIS] is the transcription initiation site (TIS), [mod-5′-UTR] comprises a modified B. subtilis 5′-UTR nucleic acid sequence, [tss codon] is a three (3) nucleotide translation start site (tss) codon, [5′-HR] is a 5′-nucleic acid sequence homology region and [3′-HR] is a 3′-nucleic acid sequence homology region, wherein the 5′-HR and 3′-HR comprise sufficient homology to a genomic (chromosomal) region (locus) immediately upstream (5′) of the [TIS ] sequence and immediately downstream (3′) of the [tss codon] sequence, respectively, to effect integration of the introduced polynucleotide construct into the genome of the modified Bacillus cell by homologous recombination. In certain embodiments, a vector or DNA expression construct comprises the polynucleotide of Formula (III). In particular embodiments, a Bacillus sp. cell comprising the polynucleotide. In particular embodiments, the Bacillus sp. cell is a Bacillus licheniformis cell.

In yet other embodiments, an open reading frame (ORF) nucleic acid sequence of the disclosure encodes a protein of interest (POI), wherein the POI is selected from the group consisting of acetyl esterases, aryl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, 62 -glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.

In yet other embodiments, the disclosure is related to an isolated polynucleotide comprising a modified-5′-UTR (mod-5′-UTR) nucleic acid sequence derived from a wild-type Bacillus sp. 5′-UTR (WT-5′-UTR) sequence, the isolated polynucleotide comprising in the 5′ to 3′ direction and operable combination the nucleic acid sequences of Formula (IV):

[TIS][5′-UTR -ΔxN][tss codon], (IV):

wherein [TIS] is the transcription initiation site (TIS), [tss codon] is a three (3) nucleotide translation start site (tss) codon and [5′-UTR -ΔxN] is a modified Bacillus sp. 5′ UTR nucleic acid sequence derived from a wild-type Bacillus sp. 5′-UTR nucleic acid sequence, wherein the mod-5′-UTR nucleic acid sequence [5′-UTR-ΔxN] comprises a deletion (−Δ) of “x” nucleotides (“N”) at the distal (3′) end of the WT-5′-UTR nucleic acid sequence.

In another embodiment, the disclosure is related to an isolated polynucleotide comprising a modified-5′-UTR (mod-5′-UTR) nucleic acid sequence derived from a wild-type Bacillus sp. 5′-UTR (WT-5′-UTR) sequence, the isolated polynucleotide comprising in the 5′ to 3′ direction and operable combination the nucleic acid sequences of Formula (V):

[TIS][5′-UTR +ΔxN][tss codon], (V):

wherein [TIS] is the transcription initiation site, [tss codon] is a three (3) nucleotide translation start site (tss) codon and [5′-UTR+ΔxN] is a modified Bacillus sp. 5′-UTR nucleic acid sequence derived from a wild-type Bacillus sp. 5′-UTR nucleic acid sequence, wherein the mod-5′-UTR nucleic acid sequence [5′-UTR+ΔxN] comprises an addition (+Δ) of “x” nucleotides (“N”) at the distal (3′) end of the wild-type Bacillus sp. 5′-UTR nucleic acid sequence.

In other embodiments, the disclosure is directed to a modified Bacillus sp. (daughter) cell producing an increased amount of a heterologous protein of interest (POI) when cultivated in a medium suitable for the production of a heterologous POI, the modified Bacillus cell comprising an introduced expression construct comprising nucleic acid sequences of Formula (I) in the 5′ to 3′ direction and in operable combination,

[Pro][mod-5′-UTR][ORF];

wherein [Pro] is a promoter region nucleic acid sequence operable in a Bacillus sp. cell, [mod-5′-UTR] is a modified B. subtilis untranslated region (mod-5′-UTR) nucleic acid sequence and [ORF] is an open reading frame nucleic acid sequence encoding a protein of interest (POI), wherein the [Pro], [mod-5′-UTR] and [ORF] nucleic acid sequences are operably linked. wherein the modified Bacillus (daughter) cell produces an increased amount of the heterologous POI relative to an unmodified Bacillus (parental) cell producing the same POI, when cultivated under similar conditions. In certain embodiments, the mod-5′-UTR comprises SEQ ID NO: 2. In other embodiments, the cell is a Bacillus licheniformis cell. In other embodiments, the ORF sequence encodes a POI selected from the group consisting of acetyl esterases, aryl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof

In certain other embodiments, the disclosure is related to a method for producing an increased amount of a heterologous protein of interest (POI) in a modified Bacillus cell comprising: (a) introducing into a parental Bacillus sp. cell an expression construct comprising in the 5′ to 3′ direction and in operable combination, nucleic acid sequences [Pro] [mod-5′-UTR] [ORF], wherein [Pro] is a promoter region nucleic acid sequence operable in a Bacillus sp. cell, [mod-5′-UTR] is a mod-5′-UTR nucleic acid sequence of SEQ ID NO: 2 and [ORF] is an open reading frame nucleic acid sequence encoding a protein of interest (POI), and (b) cultivating the modified Bacillus sp. cell of step (a) in a medium suitable for the production of a heterologous POI, wherein the modified Bacillus (daughter) cell produces an increased amount of the POI relative to a Bacillus control cell cultivated in the same medium of step (b), wherein the Bacillus control cell comprises an introduced expression construct comprising in the 5′ to 3′ direction and in operable combination nucleic acid sequences [Pro] [WT-5′-UTR] [ORF], wherein the [Pro] and [ORF] nucleic acid sequences are identical to the [Pro] and [ORF] sequence in step (a) and the [WT-5′-UTR] comprises SEQ ID NO: 1. In particular embodiments, the Bacillus cell is a Bacillus licheniformis cell. In another embodiment, the ORF sequence encodes a POI selected from the group consisting of acetyl esterases, aryl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.

In yet other embodiments, the disclosure is related to a method for producing an increased amount of an endogenous protein of interest (POI) in a modified Bacillus cell comprising: (a) obtaining a parental Bacillus cell producing an endogenous POI, (b) introducing into the cell of step (a) a polynucleotide construct comprising nucleic acid sequences of Formula (VI) in the 5′ to 3′ direction and in operable combination,

[5′-HR][mod-5′-UTR][3′-HR], (VI):

wherein [mod-5′-UTR] comprises SEQ ID NO: 2, [5′-HR] is a 5′-nucleic acid sequence homology region comprising homology to the genomic locus immediately upstream (5′) of the endogenous wild-type 5′-UTR (WT-5′-UTR) sequence of the endogenous GOI encoding the endogenous POI and [3′-HR] is a 3′-nucleic acid sequence homology region comprising homology to the genomic locus immediately downstream (3′) of the endogenous WT-5′-UTR sequence of the endogenous GOI encoding the endogenous POI, wherein the 5′-HR and 3′-HR comprise sufficient homology to said genomic loci to effect integration of the introduced mod-5′-UTR polynucleotide construct into the genome of the modified Bacillus cell by homologous recombination, thereby replacing the endogenous WT-5′-UTR with the mod-5′-UTR of SEQ ID NO: 2, and (c) cultivating the modified Bacillus sp. cell of step (b) in a medium suitable for the production of the endogenous POI, wherein the modified cell of step (c) produces an increased amount of the endogenous POI relative to the parental cell of step (a) when cultivated under similar conditions.

### BRIEF DESCRIPTION OF DRAWINGS

FIG. 1 presents a nucleic acid sequence comparison of the promoter-WT 5′ UTR-start codon nucleic acid sequence (top sequence, labelled “1”) vis-à-vis the promoter-mod 5′ UTR-start codon nucleic acid sequence (bottom sequence, labelled “2”). For both sequences, the -35 region is underlined, the −10 region is boxed, the transcription start site is labelled with “+1”, the 5′ UTR sequence is in bold characters and the ribosome binding site (RBS) is in bold italic. Vertical bars indicate identity between the two sequences. Sequence “1” is the original sequence containing the WT-5′ UTR of the B. subtilis aprE gene. Sequence “2” contains the modified aprE 5′ UTR (mod-5′ UTR), with -1A (A adenine) altering the spacing between the RBS and the start codon (ATG).

### BRIEF DESCRIPTION OF THE BIOLOGICAL SEQUENCES

SEQ ID NO: 1 is a nucleic acid sequence of a wild-type B. subtilis aprE 5′ UTR (hereinafter, “WT-5′-UTR”).

SEQ ID NO: 2 is a nucleic acid sequence of a modified B. subtilis aprE 5′ UTR (hereinafter, “mod-5′-UTR”).

SEQ ID NO: 3 is an artificial nucleic acid sequence encoding a comK transcription factor protein comprising an amino acid sequence of SEQ ID NO: 21.

SEQ ID NO: 4 is an artificial nucleic acid sequence comprising the “WT-5′-UTR” expression construct.

SEQ ID NO: 5 is an artificial nucleic acid sequence comprising the “mod-5′-UTR” expression construct.

SEQ ID NO: 6 is an artificial 5′ homology arm (i.e., 5′-HR) nucleic acid sequence comprising sequence homology to a 5′ catH gene sequence of a Bacillus licheniformis cell.

SEQ ID NO: 7 is an artificial nucleic acid sequence comprising a catH gene.

SEQ ID NO: 8 is an artificial nucleic acid sequence comprising a spoVGrrnIp hybrid promoter.

SEQ ID NO: 9 is a nucleic acid sequence encoding a B. licheniformis α-amylase protein signal sequence.

SEQ ID NO: 10 is an artificial nucleic acid sequence encoding a G. stearothermophilus variant α-amylase protein of SEQ ID NO: 13.

SEQ ID NO: 11 is a nucleic acid sequence comprising a B. licheniformis α-amylase terminator sequence.

SEQ ID NO: 12 is an artificial 3′ homology arm (i.e., 3′-HR) nucleic acid sequence comprising sequence homology to a 3′ catH gene sequence of a Bacillus licheniformis cell.

SEQ ID NO: 13 is an amino acid sequence of a variant G. stearothermophilus α-amylase protein.

SEQ ID NO: 14 is an artificial nucleic acid sequence—colony PCR “WT-5′ UTR” construct

SEQ ID NO: 15 is an artificial nucleic acid sequence—colony PCR (-1A 5′ UTR) “mod-5′ UTR” construct

SEQ ID NO: 16 is an artificial primer nucleic acid sequence.

SEQ ID NO: 17 is an artificial primer nucleic acid sequence.

SEQ ID NO: 18 is an artificial primer nucleic acid sequence.

SEQ ID NO: 19 is an artificial primer nucleic acid sequence.

SEQ ID NO: 20 is an artificial primer nucleic acid sequence.

SEQ ID NO: 21 is the amino acid sequence of the comK protein encoded by SEQ ID NO: 3.

### DETAILED DESCRIPTION

The instant disclosure is generally related to compositions and methods for producing and constructing Bacillus (host) cells (e.g., protein production host cells, cell factories) having increased protein production capabilities and the like. Certain embodiments of the disclosure are related to isolated polynucleotides comprising modified Bacillus subtilis aprE 5′-untranslated region (5′-UTR) nucleic acid sequences, vectors thereof, DNA (expression) constructs thereof, modified Bacillus (daughter) cells thereof, and methods of making and using the same. In other embodiments, the disclosure is related to isolated polynucleotides comprising a modified B. subtilis aprE 5′-untranslated region (5′-UTR) nucleic acid sequence. In certain embodiments, a modified 5′-UTR of the disclosure further comprises an upstream (5′) promoter region nucleic acid sequence which is 5′ and operably linked to the modified 5′-UTR and/or a downstream (3′) open reading frame (ORF) nucleic acid sequence (encoding a protein of interest) which is 3′ and operably linked to the modified 5′-UTR.

In another embodiment, the disclosure is directed to an isolated polynucleotide comprising Formula (I) in the 5′ to 3′ direction:

[Pro][mod-5′-UTR][ORF]; (I):

wherein [Pro] is a promoter region nucleic acid sequence operable in a Bacillus sp. cell, [mod-5′-UTR] is a modified 5′-UTR nucleic acid sequence and [ORF] is an open reading frame nucleic acid sequence encoding a protein of interest (POI), wherein the [Pro], [5′-UTR] and [ORF] nucleic acid sequences are operably linked. In other embodiments, the disclosure is related vectors and DNA constructs comprising isolated polynucleotides of the disclosure.

In other embodiments, an ORF sequence of the disclosure encodes a POI selected from the group consisting of acetyl esterases, aryl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.

In other embodiments, the disclosure is related to a modified Bacillus sp. (daughter) cell producing an increased amount of a heterologous protein of interest (POI) when cultivated in a medium suitable for the production of a heterologous POI, the modified Bacillus cell comprising an introduced expression construct comprising nucleic acid sequences of Formula (I), wherein the modified Bacillus (daughter) cell produces an increased amount of the heterologous POI relative to an unmodified Bacillus (parental) cell producing the same POI, when cultivated under similar conditions.

### I. Definitions

In view of the Bacillus strains and host cells of the disclosure, including but not limited to, (parental) Bacillus cells, modified Bacillus (daughter) cells, compositions thereof and methods of making and using the same, as described herein, the following terms and phrases are defined.

Unless otherwise indicated herein, one or more Bacillus strains (i.e., host cells) described herein can be made and used via conventional techniques commonly used in molecular biology, microbiology, protein purification, protein and DNA sequencing and various recombinant DNA methods/techniques.

Unless defined otherwise herein, all technical and scientific terms used herein have the same meaning as commonly understood by one of ordinary skill in the art.

Any definitions provided herein are to be interpreted in the context of the specification as a whole. As used herein, the singular “a,” “an” and “the” includes the plural unless the context clearly indicates otherwise. Unless otherwise indicated, nucleic acid sequences are written left to right in 5′ to 3′ orientation; and amino acid sequences are written left to right in amino to carboxy orientation. Each numerical range used herein includes every narrower numerical range that falls within such broader numerical range, as if such narrower numerical ranges were all expressly written herein.

As used herein in connection with a numerical value, the term “about” refers to a range of +/−0.5 of the numerical value, unless the term is otherwise specifically defined in context. For instance, the phrase a “pH value of about 6” refers to pH values of from 5.5 to 6.5, unless the pH value is specifically defined otherwise.

Although any methods and materials similar or equivalent to those described herein can also be used in the practice or testing of the present compositions and methods, representative illustrative methods and materials are now described. All publications and patents cited herein are incorporated by reference in their entirety.

It is further noted that the claims may be drafted to exclude any optional element. As such, this statement is intended to serve as antecedent basis for use of such exclusive terminology as “solely,” “only”, “excluding”, “not including” and the like, in connection with the recitation of claim elements, or use of a “negative” limitation or proviso thereof.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

As used herein, “the genus Bacillus” includes all species within the genus “Bacillus” as known to those of skill in the art, including but not limited to, B. subtilis, B. licheniformis, B. lentus, B. brevis, B. stearothermophilus, B. alkalophilus, B. amyloliquefaciens, B. clausii, B. halodurans, B. megaterium, B. coagulans, B. circulars, B. gibsonii, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus”, or B. polymyxa, which is now “Paenibacillus polymyxa”. The production of resistant endospores under stressful environmental conditions is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

As used herein, the phrase “untranslated region” may be abbreviated “UTR”.

As used herein the phrases “five prime untranslated region” or “5′ untranslated region” may be abbreviated as “5′-UTR” or “5′ UTR” and the phrases “three prime untranslated region” or “3′ untranslated region” may be abbreviated as “3′-UTR” or “3′ UTR”.

As used herein, a “wild-type” B. subtilis “aprE 5′ UTR” comprises a nucleotide sequence of SEQ ID NO: 1, referred to herein as “WT-5′ UTR” sequence.

As used herein, a “modified” B. subtilis “aprE 5′ UTR” or a “modified aprE 5′ UTR” are used interchangeably, and abbreviated herein as “mod-5′ UTR”.

As used herein, a “mod-5′-UTR” of the disclosure differs from a “WT-5′-UTR” (i.e., a WT-5′-UTR comprising SEQ ID NO: 1), in that the mod-5′-UTR comprises either (i) a deletion of at least the most 3′ adenine (A) nucleotide of SEQ ID NO: 1 or (ii) an addition of at least one nucleotide following the 3′ adenine (A) nucleotide of SEQ ID NO: 1.

For example, a mod-5′-UTR comprising a deletion of the most 3′ adenine nucleotide position (e.g., see SEQ ID NO: 1), may be generically represented using the following nomenclature “⁻1:mod-5′-UTR”, a mod-5′-UTR comprising a deletion of two of the most 3′ nucleotides (e.g., an adenine and guanine in SEQ ID NO: 1), may be generically represented using the following nomenclature “⁻2:mod-5′-UTR”, etc.

Likewise, a mod-5′-UTR comprising an addition of a nucleotide at the most 3′ nucleotide position (e.g., a nucleotide added 3′ to the adenine (A) in SEQ ID NO: 1), may be generically represented using the following nomenclature “⁺1:mod-5′-UTR”, a mod-5′-UTR comprising an addition of two nucleotides to the most 3′ nucleotide position (e.g., two nucleotides added 3′ to the adenine (A) in SEQ ID NO: 1), may be generically represented using the following nomenclature “⁺2:mod-5′-UTR”, etc.

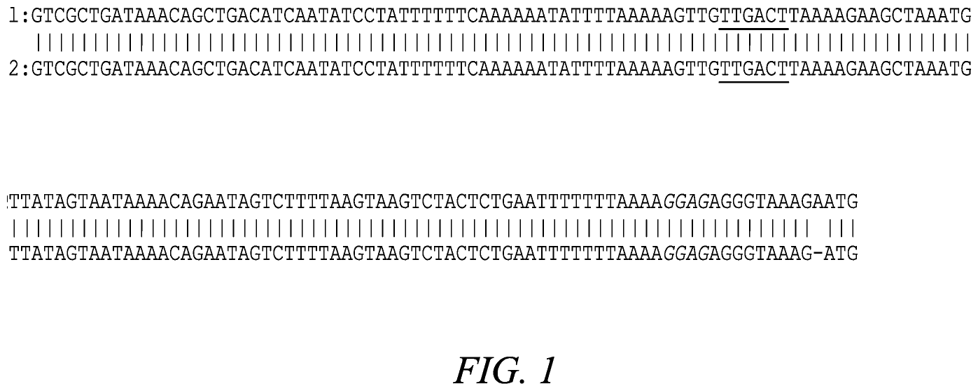

Thus, “⁻1A:mod-5′-UTR” as used herein comprises a nucleic acid sequence of SEQ ID NO: 2. Comparison of the WT-5′ UTR nucleic acid sequence (SEQ ID NO: 1) vis-à-vis the ⁻1A:mod-5′-UTR nucleic acid sequence (SEQ ID NO: 2) (e.g., see FIG. 1), shows that the ⁻1A:mod-5′-UTR sequence, relative to the WT-5′ UTR sequence, comprises a deletion of the last (3′) nucleotide (i.e., adenine (A)).

As used herein, “transcription initiation site”, abbreviated herein as “TIS”, generally refers to the base pair where transcription initiates (i.e., the start site). By convention, the transcription initiation site (TIS) in the DNA sequence of a transcription unit is usually numbered “+1”. Base pairs extending in the direction of transcription (i.e., 3′; downstream) are assigned positive “(+)” numbers, and those extending in the opposite direction (i.e., 5′; upstream) are assigned negative “(−)” numbers.

As used herein, the phrases “translation start site” and “translation start site codon” may be used interchangeably, and refer to a three (3) nucleotide translation start site (tss) codon. For example, a prokaryotic “tss codon” includes, but is not limited to, “AUG”, “GUG”, “UGG” and the like. Bioinformatics programs/tools are readily available for identifying alternate (less frequently used) start codons when searching for protein coding genes.

As used herein, a “genetically modified cell”, a “modified cell”, a “modified Bacillus cell”, a “modified cell” and the like are used interchangeably, and refer to a recombinant Bacillus cell that comprises at least one genetic modification which is not present in an unmodified Bacillus (parental) cell, from which the modified B. licheniformis (daughter) cell is derived.

As used herein, the terms “modification”, “genetic modification”, “genetic alteration”, “genetic manipulation” and the like are used interchangeably and include, but are not limited to: (a) the introduction, substitution, or removal of one or more nucleotides in a gene (or an open reading frame (ORF) thereof), or the introduction, substitution, or removal of one or more nucleotides in a regulatory element required for the transcription or translation of the gene (or ORF thereof), (b) a gene disruption, (c) a gene conversion, (d) a gene deletion, (e) a down-regulation of a gene, (f) an up-regulation of a gene, (g) specific mutagenesis and/or (h) random mutagenesis of any one or more the nucleic acid sequence or genes disclosed herein.

As used herein, “disruption of a gene”, “gene disruption”, “inactivation of a gene” and “gene inactivation” are used interchangeably and refer broadly to any genetic modification that substantially prevents a host cell from producing a functional gene product (e.g., a protein). Exemplary methods of gene disruptions include the complete or partial deletion of any portion of a gene, including a polypeptide-coding sequence (e.g., an ORF), a promoter sequence, an enhancer sequence, or another regulatory element sequence, or mutagenesis of the same, where mutagenesis encompasses substitutions, insertions, deletions, inversions, and any combinations and variations thereof which disrupt/inactivate the target gene(s) and substantially reduce or prevent the production of the functional gene product (i.e., a protein).

As used herein, the terms “down-regulation” of gene expression and “up-regulation” of gene expression include any method that results in lower (down-regulated) or higher (up-regulated) expression of a given gene. For example, the down-regulation of a gene can be achieved by RNA-induced gene silencing, genetic modifications of control elements (e.g., such as the promoter, ribosomal binding site (RBS)/Shine-Dalgarno sequences, untranslated regions (UTRs)), codon changes, and the like.

As used herein, “host cell” refers to a cell that has the capacity to act as a host or expression vehicle for a newly introduced DNA sequence (e.g., such as a vector/DNA construct). In certain embodiments, a host cells of the disclosure is a member of the genus Bacillus.

As defined herein, the terms “increased expression”, “enhanced expression”, “increased expression of a protein of interest (POI)”, “increased production”, “increased production of a POI” and the like refer to a “modified” Bacillus (daughter) cell derived from an unmodified Bacillus (parental) cell, wherein the “increase” is relative (vis-à-vis) to the “unmodified” Bacillus (parental) cell expressing/producing the same POI, when cultivated (grown, fermented) under similar conditions.

As used herein, the term “expression” refers to the transcription and stable accumulation of sense (messenger RNA, mRNA) or anti-sense RNA, derived from a nucleic acid molecule of the disclosure. Expression may also refer to translation of mRNA into a polypeptide. Thus, the term “expression” includes any step involved in the production of the polypeptide including, but not limited to, transcription, post-transcriptional modification, translation, post-translational modification, secretion and the like.

As defined herein, the combined term “expresses/produces”, as used in phrases such as a “modified cell expresses/produces an increased amount of a protein of interest, relative to an (unmodified) parental cell”, the term (“expresses/produces”) is meant to include any steps involved in the expression and production of a protein of interest in a Bacillus (host) cell of the disclosure.

As used herein, “increasing” protein production or “increased” protein production is meant an increased amount of protein produced (e.g., a protein of interest). The protein may be produced inside the cell, or secreted (or transported) into the culture medium. In certain embodiments, the protein of interest is produced (secreted) into the culture medium. Increased protein production may be detected for example, as higher maximal level of protein or enzymatic activity (e.g., such as protease activity, amylase activity, cellulase activity, hemicellulase activity and the like), or total extracellular protein produced as compared to an unmodified (parental) cell.

As used herein, “nucleic acid” refers to a nucleotide or polynucleotide sequence, and fragments or portions thereof, as well as to DNA, cDNA, and RNA of genomic or synthetic origin, which may be double-stranded or single-stranded, whether representing the sense or antisense strand. It will be understood that as a result of the degeneracy of the genetic code, a multitude of nucleotide sequences may encode a given protein. It is understood that the polynucleotides (or nucleic acid molecules) described herein include “genes”, “open reading frames” (ORFs), “vectors” and “plasmids”.

Accordingly, the term “gene”, refers to a polynucleotide that codes for a particular sequence of amino acids, which comprise all, or part of a protein coding sequence, and may include regulatory (non-transcribed) DNA sequences, such as promoter sequences, which determine for example the conditions under which the gene is expressed. The transcribed region of the gene may include untranslated regions (UTRs), including introns, 5′-untranslated regions (5′-UTRs), and 3′-untranslated regions (3′-UTRs), as well as the protein coding sequence.

As used herein, the term “coding sequence” refers to a nucleotide sequence, which directly specifies the amino acid sequence of its (encoded) protein product. The boundaries of the coding sequence are generally determined by an open reading frame, which usually begins with an “ATG” start codon. The coding sequence typically includes DNA, cDNA, and recombinant nucleotide sequences.

As used herein, the term “open reading frame” (hereinafter, “ORF”) means a nucleic acid or nucleic acid sequence (whether naturally occurring, non-naturally occurring, or synthetic) comprising an uninterrupted reading frame consisting of (i) an initiation codon, (ii) a series of two (2) or more codons representing amino acids, and (iii) a termination codon, the ORF being read (or translated) in the 5′ to 3′ direction.

The term “promoter” as used herein refers to a nucleic acid sequence capable of controlling the expression of a coding sequence or functional RNA. In general, a coding sequence is located 3′ (downstream) to a promoter sequence. Promoters may be derived in their entirety from a native gene, or be composed of different elements derived from different promoters found in nature, or comprise synthetic nucleic acid segments. It is understood by those skilled in the art that different promoters may direct the expression of a gene in different cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity. In certain embodiments, a promoter nucleic acid sequence of the disclosure comprises a promoter sequence functional in Bacillus cells, which promoter sequence includes, nut is not limited to, a low, medium or high activity constitutive promoter, an inducible promoter, a tandem promoter, a synthetic promoter, a tandem synthetic promoter, etc.

As used herein, the term “operably linked” refers to the association of nucleic acid sequences on a single nucleic acid fragment, so that the function of one is affected by the other. For example, a promoter is operably linked with a coding sequence (e.g., an ORF) when it is capable of affecting the expression of that coding sequence (i.e., the coding sequence is under the transcriptional control of the promoter). Coding sequences can be operably linked to regulatory sequences in sense or antisense orientation.

A nucleic acid is “operably linked” when it is placed into a functional relationship with another nucleic acid sequence. For example, DNA encoding a secretory leader (i.e., a signal peptide, a signal sequence), is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

For example, in certain embodiments, an isolated polynucleotide of the disclosure comprises a modified aprE 5′-UTR (mod-5′-UTR) nucleic acid sequence derived from a wild-type B. subtilis aprE 5′-UTR nucleic acid sequence (i.e., WT-5′-UTR; SEQ ID NO: 1) (e.g., see, FIG. 1).

As used herein, a “functional promoter sequence” controlling the expression of a gene of interest (or an ORF thereof) linked to the gene of interest's protein coding sequence refers to a promoter sequence which controls the transcription and translation of the coding sequence in a Bacillus cell. In certain embodiments, the functional promoter sequence used is the native promoter nucleic acid sequence as associated with the wild-type (native) gene as isolated in nature. In other embodiments, a functional promoter sequence used is a heterologous promoter nucleic acid sequence which is not associated with the wild-type (native) gene as isolated in nature, wherein the heterologous promoter is a constative promoter or an inducible promoter.

As defined herein, “suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, RNA processing site, effector binding site and stem-loop structure.

As defined herein, the term “introducing”, as used in phrases such as “introducing into a bacterial cell” or “introducing into a Bacillus cell” at least one polynucleotide open reading frame (ORF), or a gene thereof, or a vector/DNA construct thereof, includes methods known in the art for introducing polynucleotides into a cell, including, but not limited to, protoplast fusion, natural or artificial transformation (e.g., calcium chloride, electroporation), transduction, transfection, conjugation and the like (e.g., see Ferrari et al., 1989).

As used herein, “transformed” means a cell has been transformed by use of recombinant DNA techniques. Transformation typically occurs by insertion of one or more nucleotide sequences (e.g., a polynucleotide, an ORF or gene) into a cell. The inserted nucleotide sequence may be a heterologous nucleotide sequence (i.e., a sequence that is not naturally occurring in cell that is to be transformed). For example, in certain embodiments, a parental Bacillus cell is modified (e.g., transformed) by introducing into the parental cell one or more DNA constructs of the disclosure.

As used herein, “transformation” refers to introducing an exogenous DNA into a host cell so that the DNA is maintained as a chromosomal integrant or a self-replicating extra-chromosomal vector.

As used herein, “transforming DNA”, “transforming sequence”, and “DNA construct” refer to DNA that is used to introduce sequences into a host cell or organism. Transforming DNA is DNA used to introduce sequences into a host cell or organism. The DNA may be generated in vitro by PCR or any other suitable techniques. In some embodiments, the transforming DNA comprises an incoming sequence, while in other preferred embodiments it further comprises an incoming sequence flanked by homology regions (HRs). In yet a further embodiment, the transforming DNA comprises other non-homologous sequences, added to the ends (i.e., stuffer sequences or flanks). The ends can be closed such that the transforming DNA forms a closed circle, such as, for example, insertion into a vector.

As used herein, a “homology region” (abbreviated “HR”) such as a “5′-HR” or a “3′-HR” disclosed herein, refers to a nucleic acid sequence, which is homologous to a sequence in the Bacillus chromosome. More specifically, a homology region (HR) is an upstream or downstream region having between about 80 and 100% sequence identity, between about 90 and 100% sequence identity, or between about 95 and 100% sequence identity with the immediate flanking coding region of a gene, or part of a gene to be deleted, disrupted, inactivated, down-regulated and the like, according to the instant disclosure. These HR sequences direct where in the Bacillus chromosome a DNA construct is integrated, and directs what part of the Bacillus chromosome is replaced by the incoming sequence. Thus, in certain embodiments, an incoming sequence is flanked by a homology region (HR) on each side. In other embodiments the incoming sequence and the HR comprise a unit that is flanked by stuffer sequence on each side. In some embodiments, a homology region (HR sequence) is present on only a single side (either 3′ or 5′), whereas in other embodiments, it is on each side of the sequence being flanked. The sequence of each homology region is therefore homologous to a sequence in the Bacillus chromosome.

As used herein, the term “stuffer sequence” refers to any extra DNA that flanks the 5′-HR and/or the 3′-HR homology regions (e.g., vector sequences).

Thus, while not meant to limit the present disclosure, a homology region (HR) may include about between 1 base pair (bp) to 200 kilobases (kb). Preferably, a homology region (HR) includes about between 1 bp and 10.0 kb; between 1 bp and 5.0 kb; between 1 bp and 2.5 kb; between 1 bp and 1.0 kb, and between 0.25 kb and 2.5 kb. A homology region may also include about 10.0 kb, 5.0 kb, 2.5 kb, 2.0 kb, 1.5 kb, 1.0 kb, 0.5 kb, 0.25 kb and 0.1 kb. For example, in some embodiments, the 5′ and 3′ ends of a selective marker (e.g., a lysA gene, a serA gene, a pyrF gene and the like) are flanked by a homology region (5′-HR / 3′-HR) wherein the homology region comprises nucleic acid sequences immediately flanking the coding region of the gene.

As used herein, the terms “selectable marker” and “selective marker” refer to a nucleic acid (e.g., a gene or ORF) capable of expression in host cell, which allows for ease of selection of those host cells containing the selectable marker vector/DNA construct. Thus, the term “selectable marker” refers to genes (or ORFs) that provide an indication that a host cell has taken up an incoming DNA construct of interest.

As defined herein, a host cell “genome”, a bacterial (host) cell “genome”, or a Bacillus (host) cell “genome” includes chromosomal and extrachromosomal genes.

As used herein, the terms “plasmid”, “vector” and “cassette” refer to extrachromosomal elements, often carrying genes which are typically not part of the central metabolism of the cell, and usually in the form of circular double-stranded DNA molecules. Such elements may be autonomously replicating sequences, genome integrating sequences, phage or nucleotide sequences, linear or circular, of a single-stranded or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

As used herein, the term “vector” refers to any nucleic acid that can be replicated (propagated) in cells and can carry new genes (or ORFs or DNA segments) into cells. Thus, the term refers to a nucleic acid construct designed for transfer between different host cells. Vectors include viruses, bacteriophage, pro-viruses, plasmids, phagemids, transposons, and artificial chromosomes such as YACs (yeast artificial chromosomes), BACs (bacterial artificial chromosomes), PLACs (plant artificial chromosomes), and the like, that are “episomes” (i.e., replicate autonomously or can integrate into a chromosome of a host organism).

As used herein, the terms “expression cassette”, “DNA expression cassette” and “expression vector” refer to a nucleic acid (DNA) construct generated recombinantly or synthetically, with a series of specified nucleic acid elements that permit transcription of a particular nucleic acid in a target cell (i.e., these are vectors or vector elements, as described above). The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In some embodiments, DNA constructs also include a series of specified nucleic acid elements that permit transcription of a particular nucleic acid in a target cell.

As used herein, a “targeting vector” is a vector that includes polynucleotide sequences that are homologous to a region in the chromosome of a host cell into which the targeting vector is transformed and that can drive homologous recombination at that region. For example, targeting vectors find use in introducing or removing mutations in the chromosome of a host cell through homologous recombination. In certain embodiments, such targeting vectors are usefully employed in inactivating one or more genes in (parental) Bacillus cells to modified Bacillus (daughter) cells thereof. For example, in certain embodiments, a targeting vector is used to inactivate Bacillus genes, Bacillus promoter sequences, Bacillus 5′-UTR sequences, Bacillus 3′-UTR sequences and the like, and combinations thereof. In some embodiments, the targeting vector comprises other non-homologous sequences, e.g., added to the ends (i.e., stuffer sequences or flanking sequences). The ends can be closed such that the targeting vector forms a closed circle, such as, for example, insertion into a vector. Selection and/or construction of appropriate vectors is well within the knowledge of those having skill in the art.

As used herein, the term “plasmid” refers to a circular double-stranded (ds) DNA construct used as a cloning vector, and which forms an extrachromosomal self-replicating genetic element in many bacteria and some eukaryotes. In some embodiments, plasmids become incorporated into the genome of the host cell.

As used herein, the term “protein of interest” or “POI” refers to a polypeptide of interest that is desired to be expressed in a Bacillus cell, particularly a modified Bacillus cell, wherein the POI is preferably expressed at increased levels (i.e., relative to the “unmodified” Bacillus (parental) cell). Thus, as used herein, a POI may be an enzyme, a substrate-binding protein, a surface-active protein, a structural protein, a receptor protein, and the like. In certain embodiments, a modified cell of the disclosure produces an increased amount of a heterologous protein of interest or an endogenous protein of interest, relative to an unmodified Bacillus (parental) cell. In particular embodiments, an increased amount of a protein of interest produced by a modified cell of the disclosure is at least a 0.5% increase, at least a 1.0% increase, at least a 5.0% increase, or a greater than 5.0% increase, relative to the parental cell. In certain embodiments, an increased amount is by determined enzymatic activity of the encoded POI, changes in mRNA, optical density measurements, protein binding assays, and the like.

As defined herein, a “gene of interest” or “GOI” refers a nucleic acid sequence (e.g., a polynucleotide, a gene or an ORF) which encodes a POI. A “gene of interest” encoding a “protein of interest” may be a naturally occurring gene, a mutated gene or a synthetic gene.

As used herein, the terms “polypeptide” and “protein” are used interchangeably, and refer to polymers of any length comprising amino acid residues linked by peptide bonds. The conventional one (1) letter or three (3) letter codes for amino acid residues are used herein. The polypeptide may be linear or branched, it may comprise modified amino acids, and it may be interrupted by non-amino acids. The term polypeptide also encompasses an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

In certain embodiments, a gene of the instant disclosure encodes a commercially relevant industrial protein of interest, such as an enzyme (e.g., acetyl esterases, aryl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α- glucosidases, β-glucosidases, glucuronidases, glycosyl hydrolases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, DNases, transferases, transport proteins, transglutaminases, xylanases, hexose oxidases, and combinations thereof).

As used herein, a “variant” polypeptide refers to a polypeptide that is derived from a parent (or reference) polypeptide by the substitution, addition, or deletion of one or more amino acids, typically by recombinant DNA techniques. Variant polypeptides may differ from a parent polypeptide by a small number of amino acid residues and may be defined by their level of primary amino acid sequence homology/identity with a parent (reference) polypeptide.

Preferably, variant polypeptides have at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity with a parent (reference) polypeptide sequence. As used herein, a “variant” polynucleotide refers to a polynucleotide encoding a variant polypeptide, wherein the “variant polynucleotide” has a specified degree of sequence homology/identity with a parent polynucleotide, or hybridizes with a parent polynucleotide (or a complement thereof) under stringent hybridization conditions. Preferably, a variant polynucleotide has at least 70%, at least 75%, at least 80%, at least 85%, at least 90%, at least 91%, at least 92%, at least 93%, at least 94%, at least 95%, at least 96%, at least 97%, at least 98%, or even at least 99% nucleotide sequence identity with a parent (reference) polynucleotide sequence.

As used herein, a “mutation” refers to any change or alteration in a nucleic acid sequence. Several types of mutations exist, including point mutations, deletion mutations, silent mutations, frame shift mutations, splicing mutations and the like. Mutations may be performed specifically (e.g., via site directed mutagenesis) or randomly (e.g., via chemical agents, passage through repair minus bacterial strains).

As used herein, in the context of a polypeptide or a sequence thereof, the term “substitution” means the replacement (i.e., substitution) of one amino acid with another amino acid.

As defined herein, an “endogenous gene” refers to a gene in its natural location in the genome of an organism.

As defined herein, a “heterologous” gene, a “non-endogenous” gene, or a “foreign” gene refer to a gene (or ORF) not normally found in the host organism, but that is introduced into the host organism by gene transfer.

As defined herein, a “heterologous nucleic acid construct” or a “heterologous nucleic acid sequence” has a portion of the sequence which is not native to the cell in which it is expressed.

The term “derived” encompasses the terms “originated”, “obtained”, “obtainable” and “created,” and generally indicates that one specified material or composition finds its origin in another specified material or composition, or has features that can be described with reference to the another specified material or composition.

As used herein, the term “homology” relates to homologous polynucleotides or polypeptides. If two or more polynucleotides or two or more polypeptides are homologous, this means that the homologous polynucleotides or polypeptides have a “degree of identity” of at least 60%, more preferably at least 70%, even more preferably at least 85%, still more preferably at least 90%, more preferably at least 95%, and most preferably at least 98%. Whether two polynucleotide or polypeptide sequences have a sufficiently high degree of identity to be homologous as defined herein, can suitably be investigated by aligning the two sequences using computer programs and techniques known in the art, (See e.g., Smith and Waterman, 1981; Needleman and Wunsch, 1970; Pearson and Lipman, 1988; programs such as GAP, BESTFIT, FASTA, and TFASTA in the Wisconsin Genetics Software Package (Genetics Computer Group, Madison, Wis.) and Devereux et. al., 1984).

As used herein, the term “percent (%) identity” refers to the level of nucleic acid or amino acid sequence identity between the nucleic acid sequences that encode a polypeptide or the polypeptide's amino acid sequences, when aligned using a sequence alignment program.

As used herein, “specific productivity” is total amount of protein produced per cell per time over a given time period.

As defined herein, the terms “purified”, “isolated” or “enriched” are meant that a biomolecule (e.g., a polypeptide or polynucleotide) is altered from its natural state by virtue of separating it from some, or all of, the naturally occurring constituents with which it is associated in nature. Such isolation or purification may be accomplished by art-recognized separation techniques such as ion exchange chromatography, affinity chromatography, hydrophobic separation, dialysis, protease treatment, ammonium sulphate precipitation or other protein salt precipitation, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to a purified or isolated biomolecule composition which provide additional benefits, for example, activating agents, anti-inhibition agents, desirable ions, compounds to control pH or other enzymes or chemicals.

As used herein, the term “ComK polypeptide” is defined as the product of a comK gene; a transcription factor that acts as the final auto-regulatory control switch prior to competence development; involved with activation of the expression of late competence genes involved in DNA-binding and uptake and in recombination (Liu and Zuber, 1998, Hamoen et al., 1998). In certain embodiments of the disclosure, a Bacillus cell comprises an introduced plasmid encoding the comK transcription factor. Exemplary comK nucleic acid and polypeptide sequences are set forth in SEQ ID NO: 3 and SEQ ID NO: 21, respectively.

As used herein, “homologous genes” refers to a pair of genes from different, but usually related species, which correspond to each other and which are identical or very similar to each other. The term encompasses genes that are separated by speciation (i.e., the development of new species) (e.g., orthologous genes), as well as genes that have been separated by genetic duplication (e.g., paralogous genes).

As used herein, “orthologue” and “orthologous genes” refer to genes in different species that have evolved from a common ancestral gene (i.e., a homologous gene) by speciation. Typically, orthologs retain the same function during the course of evolution. Identification of orthologs finds use in the reliable prediction of gene function in newly sequenced genomes.

As used herein, “paralog” and “paralogous genes” refer to genes that are related by duplication within a genome. While orthologs retain the same function through the course of evolution, paralogs evolve new functions, even though some functions are often related to the original one. Examples of paralogous genes include, but are not limited to genes encoding trypsin, chymotrypsin, elastase, and thrombin, which are all serine proteinases and occur together within the same species.

As used herein, an “analogous sequence” is one wherein the function of the gene is essentially the same as the gene derived from a B. licheniformis cell. Additionally, analogous genes include at least 60%, 65%, 70%, 75%, 80%, 85%, 90%, 95%, 97%, 98%, 99% or 100% sequence identity with the sequence of the Bacillus licheniformis cell. Analogous sequences are determined by known methods of sequence alignment. A commonly used alignment method is BLAST, although there are other methods that also find use in aligning sequences.

As used herein, the term “hybridization” refers to the process by which a strand of nucleic acid joins with a complementary strand through base pairing, as known in the art. A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tₘ) of the nucleic acid binding complex or probe. For example, “maximum stringency” typically occurs at about Tₘ⁻5° C. (5° below the Tₘ of the probe); “high stringency” at about 5-10° C. below the Tₘ; “intermediate stringency” at about 10-20° C. below the Tₘ of the probe; and “low stringency” at about 20-25° C. below the Tₘ. Functionally, maximum stringency conditions may be used to identify sequences having strict identity or near-strict identity with the hybridization probe; while an intermediate or low stringency hybridization can be used to identify or detect polynucleotide sequence homologs. Moderate and high stringency hybridization conditions are well known in the art. An example of high stringency conditions includes hybridization at about 42° C. in 50% formamide, 5× SSC, 5× Denhardt's solution, 0.5% SDS and 100 pg/ml denatured carrier DNA, followed by washing two times in 2× SSC and 0.5% SDS at room temperature (RT) and two additional times in 0. 1× SSC and 0.5% SDS at 42° C. An example of moderate stringent conditions including overnight incubation at 37° C. in a solution comprising 20% formamide, 5 ×SSC (150mM NaCl, 15 mM trisodium citrate), 50 mM sodium phosphate (pH 7.6), 5 x Denhardt's solution, 10% dextran sulfate and 20 mg/ml denaturated sheared salmon sperm DNA, followed by washing the filters in 1× SSC at about 37-50° C. Those of skill in the art know how to adjust the temperature, ionic strength, etc. as necessary to accommodate factors such as probe length and the like.

As used herein, “recombinant” includes reference to a cell or vector, that has been modified by the introduction of a heterologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention. “Recombination”, “recombining” or generating a “recombined” nucleic acid is generally the assembly of two or more nucleic acid fragments wherein the assembly gives rise to a chimeric gene.

### II. Modified 5′-UTR Sequences for Increased Protein Production in Bacillus Host Cells

The instant disclosure is generally related to compositions and methods for producing and constructing Bacillus (host) cells (e.g., protein production host cells, cell factories) having increased protein production capabilities and the like. Certain embodiments of the disclosure are therefore related to isolated polynucleotides comprising modified B. subtilis aprE 5′-untranslated region (mod-5′-UTR) nucleic acid sequences, vectors thereof, DNA (expression) constructs thereof, modified Bacillus (daughter) cells thereof, and methods of making and using the same.

For example, it is generally understood in the art, that messenger RNA (mRNA) translation “initiation” is fundamentally important for all protein-coding genes in the genomes of all organisms. More particularly, “initiation”, rather than “elongation”, is usually the rate-limiting step in translation, and proceeds at very different efficiencies depending on the sequences in the 5′ UTRs of the mRNAs (Jacques & Dreyfus, 1990). For example, in prokaryotes (i.e., both eubacteria and archaebacteria), the Shine-Dalgarno (SD) sequence in a mRNA is well known as the initiator element of translation (Shine and Dalgarno, 1974; Shine and Dalgarno, 1975). The SD sequence, typically “GGAGG” is located approximately 10 nucleotides upstream (5′) of the initiator codon. The SD sequence pairs with a complementary sequence “CCUCC” in the 3′ end of a 16S rRNA. In the 16S rRNA, the complementary sequence (CCUCC) is called the anti-SD sequence in the 3′ tail of which region is single stranded. The interaction between the SD and the anti-SD sequences (the SD interaction) augments initiation by anchoring the small (30S) ribosomal subunit around the initiation codon to form a “pre-initiation complex” (Dontsova et al., 1991), wherein the importance of the SD interaction for the efficient initiation of translation has been experimentally verified for both eubacteria (e.g., Bacillus sp.) and archaebacteria (Jacob et al., 1987).

Thus, as stated briefly above, Applicants of the present disclosure have identified surprising and unexpected results related to mRNA nucleotide spacing(s) between the SD sequence (i.e., the RBS) and the translation start site [tss] codon (ATG/AUG) position. Without wishing to be bound by any particular theory, mechanism or mode of action, Applicant contemplates and presents results herein that varying the position of the SD sequence (RBS) with respect to the start codon (ATG/AUG), substantially increases the production of proteins of interest when such sequences are introduced and expressed in Bacillus cells.

More particularly, Example 1 of the disclosure is related to the design/construction of modified 5′-untranslated region (5′-UTRs) nucleic acid sequences. For example, expression cassettes were constructed comprising either the wild-type B. subtilis aprE 5′ UTR sequence (WT-5′-UTR; SEQ ID NO: 1) or a modified 5′-UTR sequence (⁻1A:mod-5′-UTR; SEQ ID NO: 2, see FIG. 1) and introduced into a parental Bacillus cells, wherein the WT-5′-UTR and ⁻1A:mod-5′-UTR were operably linked to an upstream (5′) promoter and a downstream (3′) open reading frame encoding the protein of interest (i.e., an α-amylase).

As presented in Example 2, the relative α-amylase production from Bacillus (daughter) cells comprising the WT-5′-UTR and from Bacillus (daughter) cells comprising the ⁻1A:mod-5′-UTR were measured using standard methods, wherein the daughter cells comprising the ⁻1A:mod-5′-UTR expression construct) produced 20% more α-amylase than the daughter cells comprising the WT-5′ UTR expression construct. More particularly, on a per OD550 unit basis, the daughter cells comprising the ⁻1A:mod-5′-UTR construct produced 40% more α-amylase than the daughter cells comprising the WT-5′ UTR expression construct (e.g., see, Table 1).

Thus, in certain embodiments, the disclosure is related to isolated polynucleotides comprising modified B. subtilis aprE 5′-untranslated region (mod-5′-UTR) nucleic acid sequences. In certain other embodiments, a modified 5′-UTR (mod-5′-UTR) of the disclosure further comprises an upstream (5′) promoter region nucleic acid sequence which is 5′ and operably linked to the modified 5′-UTR and/or a downstream (3′) open reading frame (ORF) nucleic acid sequence (encoding a protein of interest) which is 3′ and operably linked to the modified 5′-UTR.

In other embodiments, the disclosure is directed to isolated polynucleotides comprising Formula (I) in the 5′ to 3′ direction:

[Pro][mod-5′-UTR][ORF]; (I):

wherein [Pro] is a promoter region nucleic acid sequence operable in a Bacillus sp. cell, [mod-5′-UTR] is a modified B. subtilis aprE 5′ untranslated region (mod-5′-UTR) nucleic acid sequence and [ORF] is an open reading frame nucleic acid sequence encoding a protein of interest (POI), wherein the [Pro], [mod-5′-UTR] and [ORF] nucleic acid sequences are operably linked. In other embodiments, the disclosure is related vectors and DNA constructs comprising an isolated polynucleotide of the disclosure.

In other embodiments, the disclosure is related to modified Bacillus sp. (daughter) cell producing an increased amount of a heterologous protein of interest (POI) when cultivated in a medium suitable for the production of a heterologous POI, the modified Bacillus cell comprising an introduced expression construct comprising nucleic acid sequences of Formula (I), wherein the modified Bacillus (daughter) cell produces an increased amount of the heterologous POI relative to an unmodified Bacillus (parental) cell producing the same POI, when cultivated under similar conditions.

In other embodiments, the disclosure is related to isolated polynucleotides comprising such mod-5′-UTR nucleic acid sequences, wherein the polynucleotide comprises a mod-5′-UTR nucleic acid sequence comprising in the 5′ to 3′ direction and operably combined, the nucleic acid sequences presented in Formula (II):

[TIS][mod-5′ UTR][tss codon] (II):

wherein [TIS] is the transcription initiation site (TIS), [mod-5′-UTR] comprises a modified B. subtilis aprE 5′-UTR nucleic acid sequence and [tss codon] is a three (3) nucleotide translation start site (tss) codon.

In certain other embodiments, such “modified” aprE 5 ‘-UTR (mod-5’-UTR) nucleic acid sequences disclosed herein are related to an isolated polynucleotide comprising a nucleic acid sequences of Formula (III) in the 5′ to 3′ direction and in operable combination,

[5′-HR][TIS][mod-5′-UTR][tss codon][3′-HR], (III):

wherein [TIS] is the transcription initiation site (TIS), [mod-5′-UTR] comprises a modified B. subtilis aprE 5′-UTR nucleic acid sequence, [tss codon] is a three (3) nucleotide translation start site (tss) codon, [5′-HR] is a 5′-nucleic acid sequence homology region and [3′-HR] is a 3′-nucleic acid sequence homology region, wherein the 5′-HR and 3′-HR comprise sufficient homology to a genomic (chromosomal) region (locus) immediately upstream (5′) of the [TIS] sequence and immediately downstream (3′) of the [tss codon] sequence, respectively, to effect integration of the introduced polynucleotide construct into the genome of the modified Bacillus cell by homologous recombination.

For example, in certain embodiments, the disclosure is related to modified Bacillus (daughter) cells producing an increased amount of an endogenous POI relative to an unmodified Bacillus (parental) cell producing the same POI, when cultivated under similar conditions. Thus, in certain embodiments, a parental Bacillus sp. is modified by introducing (e.g., transforming) a polynucleotide construct of Formula (III) or the like, into a parental Bacillus cell, wherein a modified (daughter) Bacillus cell derived therefrom comprises a modified 5′ UTR (e.g., mod-5′ UTR; SEQ ID NO: 2) integrated into the targeted (chromosomal) genomic locus, as provided by the 5′-HR and 3′-HR.

Another embodiment of the disclosure is related to an isolated polynucleotide comprising a mod-5′-UTR nucleic acid sequence derived from a wild-type Bacillus sp. 5′-UTR sequence, the isolatedpolynucleotide comprising in the 5′ to 3′ direction and operable combination the nucleic acid sequences of Formula (IV):

[TIS][5′-UTR ⁻ΔxN] [tss codon], (IV):

wherein [TIS] is the transcription initiation site (TIS), [tss codon] is a three (3) nucleotide translation start site (tss) codon and [5′-UTR⁻ΔxN] is a modified Bacillus sp. 5′-UTR nucleic acid sequence derived from a wild-type Bacillus sp. 5′-UTR nucleic acid sequence, wherein the mod-5′-UTR nucleic acid sequence [5′-UTR⁻ΔxN] comprises a deletion (⁻Δ) of “x” nucleotides (“N”) at the distal (3′) end of the wild-type Bacillus sp. 5′-UTR nucleic acid sequence. For example, a mod-5′-UTR nucleic acid sequence comprising a single nucleotide deletion at the at the distal (3′) end of the wild-type Bacillus sp. 5′-UTR nucleic acid sequence, can be represented as “[5′-UTR ⁻Δ1N]”, a mod-5′-UTR nucleic acid sequence comprising two nucleotides deleted at the at the distal (3′) end of the wild-type Bacillus sp. 5′-UTR nucleic acid sequence, can be represented as “[5′-UTR ⁻Δ2N]”, etc.

Likewise, in certain other embodiments, the disclosure is related to an isolated polynucleotide comprising a mod-5′-UTR nucleic acid sequence derived from a wild-type Bacillus sp. 5′-UTR sequence, the isolated polynucleotide comprising in the 5′ to 3′ direction and operable combination the nucleic acid sequences of Formula (V):

[TIS][5′-UTR ⁺Δx/V][tss codon], (V):

wherein [TIS] is the transcription initiation site, [tss codon] is a three (3) nucleotide translation start site (tss) codon and [5′-UTR ⁺ΔxN] is a modified Bacillus sp. 5′-UTR nucleic acid sequence derived from a wild-type Bacillus sp. 5′-UTR nucleic acid sequence, wherein the mod-5′-UTR nucleic acid sequence [5′-UTR ⁺ΔxN] comprises an addition (⁺Δ) of “x” nucleotides (“N”) at the distal (3′) end of the wild-type Bacillus sp. 5′-UTR nucleic acid sequence. For example, a mod-5′-UTR nucleic acid sequence comprising a single nucleotide addition at the at the distal (3′) end of the wild-type Bacillus sp. 5′-UTR nucleic acid sequence, can be represented as “[5′-UTR ⁺Δ1N”, a mod-5′-UTR nucleic acid sequence comprising two added nucleotides at the distal (3′) end of the wild-type Bacillus sp. 5′-UTR nucleic acid sequence, can be represented as “5′-UTR⁺Δ2N]”, etc.

Thus, in certain embodiments, one or more isolated polynucleotides of the disclosure are constructed as set forth in Formula (IV) or (V), wherein the mod-5′-UTRs are designed/constructed to have progressively shorter (3′) ends (i.e., as “⁻ΔxN” increases) or progressively longer (3′) ends (as “⁺ΔxN” increases). For example, deletion of the −1 nucleotide position provides a one (1) bp reduction in the spacing between ribosomal binding site (RBS) (located within the 5′-UTR sequence) and the translation start site codon (tss codon) (e.g., see, FIG. 1).

Thus, as set forth above, certain embodiments of the disclosure are related to such modified Bacillus (daughter) cells producing an increased amount of an endogenous or heterologous POI relative to an unmodified Bacillus (parental) cell producing the same POI, when cultivated under similar conditions. Thus, certain other embodiments of the disclosure are related to wild-type (native) nucleic acid sequences, variant nucleic acid sequences, modified nucleic acid sequences, the analysis of such nucleic acid sequences and the identification of certain nucleic acid sequence features therein including, but not limited to, transcription initiation site (TIS) sequences, translation start site (tss) codons, open reading frames (ORFs), 5′-UTRs, 3′-UTRs, promoters, promoter regions, and the like.

For example, as stated in the Definitions section supra, a transcription initiation site (TIS) refers to the base pair where transcription initiates (i.e., the start site). One of skill in the art can readily identify such TIS sequences associated with a particular gene (ORF) sequence by visual analysis of the DNA sequence, and more particularly with the use of bioinformatics programs and tools which analyze an input sequence(s) for various gene regulatory elements, such as TIS sequences, promoter regions, UTRs and the like. A translation start site (tss), as previously defined supra, refers to a three (3) nucleotide translation start site (tss) codon. Exemplary prokaryotic tss codons (in order of their frequency of occurrence, high to low), include “AUG”, “GUG” and “UGG”. For example, one skilled in the art can identify promoters using a regular expression search for an identical, or near identical match to known sigma factor binding sites in the organism of interest (e.g., using data from Haldenwang et al., 1995). Once a putative promoter has been identified, a putative TIS sequence can be assigned.

Additional bioinformatics tools for identifying nucleic acid sequence features such as promoters and the like include, but are not limited to, PromoterHunter (Klucar et al., 2010), PromPredict (Bansal, 2009), BacPP (de Avila et al., 2011), BPROM (Salamov, 2011) and the PRODORIC tool which can predict binding of a number of proteins to a DNA sequence and assign a weighted probability score to each predicted promoter (Munch et al., 2003). Additionally, deep learning neural networks can be trained to effectively predict promoter sequences by training with known promoters from a given organism (Kh et al., PLOSone, Feb. 3, 2017). Once the TIS is identified, the 5′ UTR can be inferred as the sequence 3′ of and including the TIS until and excluding the first nucleotide of the TSS. Once the putative 5′ UTR is identified, further modifications of the 5′ UTR can be made as described herein.

### III. Molecular Biology

As set forth above, certain embodiments of the disclosure are related to (recombinant) genetically modified Bacillus cells derived from parental Bacillus cells. In certain embodiments, a Bacillus cell of the disclosure is genetically modified for increased expression/production of one or more proteins of interest. In particular embodiments, a Bacillus cell of the disclosure is genetically modified to expresses a gene of interest encoding a protein of interest from a DNA construct comprising a mod-5′-UTR of the disclosure. In yet other embodiments, a parental Bacillus cell is genetically modified to inactivate one or more (endogenous) chromosomal genes and/or is modified to restore one or more (endogenous) chromosomal genes which are inactive.

Thus, certain embodiments of the disclosure are generally related to compositions and methods for producing and constructing Bacillus host cells (e.g., protein production host cells, cell factories) having increased protein production capabilities. Thus, certain embodiments of disclosure are related to methods for genetically modifying cells of the disclosure, wherein the modification comprises (a) the introduction, substitution, or removal of one or more nucleotides in a gene (or an ORF thereof), or the introduction, substitution, or removal of one or more nucleotides in a regulatory element required for the transcription or translation of the gene or ORF thereof, (b) a gene disruption, (c) a gene conversion, (d) a gene deletion, (e) a gene down-regulation, (f) site specific mutagenesis and/or (g) random mutagenesis.

For example, in certain embodiments, a modified Bacillus cell of the disclosure is constructed by reducing or eliminating the expression of a gene, using methods well known in the art, for example, insertions, disruptions, replacements, or deletions. The portion of the gene to be modified or inactivated may be, for example, the coding region or a regulatory element required for expression of the coding region. An example of such a regulatory or control sequence may be a promoter sequence or a functional part thereof, (i.e., a part which is sufficient for affecting expression of the nucleic acid sequence). Other control sequences for modification include, but are not limited to, a leader sequence, a pro-peptide sequence, a signal sequence, a transcription terminator, a transcriptional activator and the like.

In certain other embodiments, a modified Bacillus cell is constructed by gene deletion to eliminate or reduce the expression of at least one gene. Gene deletion techniques enable the partial or complete removal of the gene(s), thereby eliminating their expression, or expressing a non-functional (or reduced activity) protein product. In such methods, the deletion of the gene(s) may be accomplished by homologous recombination, e.g., using a plasmid/vector that has been constructed to contiguously contain the 5′ and 3′ regions flanking (i.e., 5′-HR and 3′-HR) the gene. The contiguous 5′ and 3′ regions may be introduced into a Bacillus cell, for example, on a temperature-sensitive plasmid, such as pE194, in association with a second selectable marker at a permissive temperature to allow the plasmid to become established in the cell. The cell is then shifted to a non-permissive temperature to select for cells that have the plasmid integrated into the chromosome at one of the homologous flanking regions. Selection for integration of the plasmid is effected by selection for the second selectable marker. After integration, a recombination event at the second homologous flanking region is stimulated by shifting the cells to the permissive temperature for several generations without selection. The cells are plated to obtain single colonies and the colonies are examined for loss of both selectable markers (see, e.g., Perego, 1993). Thus, a person of skill in the art may readily identify nucleotide regions in the gene's coding sequence and/or the gene's non-coding sequence suitable for complete or partial deletion.

In other embodiments, a modified Bacillus cell of the disclosure is constructed by introducing, substituting, or removing one or more nucleotides in the gene or a regulatory element required for the transcription or translation thereof For example, nucleotides may be inserted or removed so as to result in the introduction of a stop codon, the removal of the start codon, or a frame-shift of the open reading frame. Such a modification may be accomplished by site-directed mutagenesis, or PCR generated mutagenesis, in accordance with methods known in the art (e.g., see, Botstein and Shortie, 1985; Lo et al., 1985; Higuchi et al., 1988; Shimada, 1996; Ho et al., 1989; Horton et al., 1989 and Sarkar and Sommer, 1990). Thus, in certain embodiments, a gene of the disclosure is inactivated by complete or partial deletion.

In another embodiment, a modified Bacillus cell is constructed by the process of gene conversion (e.g., see Iglesias and Trautner, 1983). For example, in the gene conversion method, a nucleic acid sequence corresponding to the gene(s) is mutagenized in vitro to produce a defective nucleic acid sequence, which is then transformed into the parental Bacillus cell to produce a defective gene. By homologous recombination, the defective nucleic acid sequence replaces the endogenous gene. It may be desirable that the defective gene or gene fragment also encodes a marker which may be used for selection of transformants containing the defective gene. For example, the defective gene may be introduced on a non-replicating or temperature-sensitive plasmid in association with a selectable marker. Selection for integration of the plasmid is effected by selection for the marker under conditions not permitting plasmid replication. Selection for a second recombination event leading to gene replacement is effected by examination of colonies for loss of the selectable marker and acquisition of the mutated gene (Perego, 1993). Alternatively, the defective nucleic acid sequence may contain an insertion, substitution, or deletion of one or more nucleotides of the gene, as described below.

In other embodiments, a modified Bacillus cell is constructed by established anti-sense techniques using a nucleotide sequence complementary to the nucleic acid sequence of the gene (Parish and Stoker, 1997). More specifically, expression of a gene by a Bacillus cell can be reduced (down-regulated) or eliminated by introducing a nucleotide sequence complementary to the nucleic acid sequence of the gene, which may be transcribed in the cell and is capable of hybridizing to the mRNA produced in the cell. Under conditions, allowing the complementary anti-sense nucleotide sequence to hybridize to the mRNA, the amount of protein translated is thus reduced or eliminated. Such anti-sense methods include, but are not limited to RNA interference (RNAi), small interfering RNA (siRNA), microRNA (miRNA), antisense oligonucleotides, Cas9 mediated gene silencing and the like, all of which are well known to the skilled artisan.

In other embodiments, a modified Bacillus cell is produced/constructed via CRISPR-Cas9 editing. For example, a gene encoding a protein of interest can be disrupted (or deleted or down-regulated) by means of nucleic acid guided endonucleases, that find their target DNA by binding either a guide RNA (e.g., Cas9) and Cpfl or a guide DNA (e.g., NgAgo), which recruits the endonuclease to the target sequence on the DNA, wherein the endonuclease can generate a single or double stranded break in the DNA. This targeted DNA break becomes a substrate for DNA repair, and can recombine with a provided editing template to disrupt or delete the gene. For example, the gene encoding the nucleic acid guided endonuclease (for this purpose Cas9 from S. pyogenes) or a codon optimized gene encoding the Cas9 nuclease is operably linked to a promoter active in the Bacillus cell and a terminator active in Bacillus cell, thereby creating a Bacillus Cas9 expression cassette. Likewise, one or more target sites unique to the gene of interest are readily identified by a person skilled in the art. For example, to build a DNA construct encoding a gRNA -directed to a target site within the gene of interest, the variable targeting domain (VT) will comprise nucleotides of the target site which are 5′ of the (PAM) proto-spacer adjacent motif, e.g., NGG for S. pyogenes Cas9, which nucleotides are fused to DNA encoding the Cas9 endonuclease recognition domain for S. pyogenes Cas9 (CER). The combination of the DNA encoding a VT domain and the DNA encoding the CER domain thereby generate a DNA encoding a gRNA. Thus, a Bacillus expression cassette for the gRNA is created by operably linking the DNA encoding the gRNA to a promoter active in Bacillus cells and a terminator active in Bacillus cells.

In certain embodiments, the DNA break induced by the endonuclease is repaired/replaced with an incoming sequence. For example, to precisely repair the DNA break generated by the Cas9 expression cassette and the gRNA expression cassette described above, a nucleotide editing template is provided, such that the DNA repair machinery of the cell can utilize the editing template. For example, about 500 bp 5′ of targeted gene can be fused to about 500 bp 3′ of the targeted gene to generate an editing template, which template is used by the Bacillus host's machinery to repair the DNA break generated by the RGEN.

The Cas9 expression cassette, the gRNA expression cassette and the editing template can be co-delivered to cells using many different methods (e.g., protoplast fusion, electroporation, natural competence, or induced competence). The transformed cells are screened by PCR amplifying the target gene locus, by amplifying the locus with a forward and reverse primer. These primers can amplify the wild-type locus or the modified locus that has been edited by the RGEN. These fragments are then sequenced using a sequencing primer to identify edited colonies.

In yet other embodiments, a modified Bacillus cell is constructed by random or specific mutagenesis using methods well known in the art, including, but not limited to, chemical mutagenesis (see, e.g., Hopwood, 1970) and transposition (see, e.g., Youngman et al., 1983). Modification of the gene may be performed by subjecting the parental cell to mutagenesis and screening for mutant cells in which expression of the gene has been reduced or eliminated. The mutagenesis, which may be specific or random, may be performed, for example, by use of a suitable physical or chemical mutagenizing agent, use of a suitable oligonucleotide, or subjecting the DNA sequence to PCR generated mutagenesis. Furthermore, the mutagenesis may be performed by use of any combination of these mutagenizing methods.

Examples of a physical or chemical mutagenizing agent suitable for the present purpose include ultraviolet (UV) irradiation, hydroxylamine, N-methyl-N′-nitro-N-nitrosoguanidine (MNNG), N-methyl-N′-nitrosoguanidine (NTG), O-methyl hydroxylamine, nitrous acid, ethyl methane sulphonate (EMS), sodium bisulphite, formic acid, and nucleotide analogues. When such agents are used, the mutagenesis is typically performed by incubating the parental cell to be mutagenized in the presence of the mutagenizing agent of choice under suitable conditions, and selecting for mutant cells exhibiting reduced or no expression of the gene.

In certain other embodiments, a modified Bacillus cell comprises a deletion of an endogenous (chromosomal) gene. In certain other embodiments, a modified Bacillus cell comprises a disruption of an endogenous (chromosomal) gene. In other embodiments, a modified Bacillus cell comprises a down-regulated endogenous (chromosomal) gene.

PCT Publication No. WO2003/083125 discloses methods for modifying Bacillus cells, such as the creation of Bacillus deletion strains and DNA constructs using PCR fusion to bypass E. coli.

PCT Publication No. WO2002/14490 discloses methods for modifying Bacillus cells including (1) the construction and transformation of an integrative plasmid (pComK), (2) random mutagenesis of coding sequences, signal sequences and pro-peptide sequences, (3) homologous recombination, (4) increasing transformation efficiency by adding non-homologous flanks to the transformation DNA, (5) optimizing double cross-over integrations, (6) site directed mutagenesis and (7) marker-less deletion.

Those of skill in the art are well aware of suitable methods for introducing polynucleotide sequences into bacterial cells (e.g., E. coli and Bacillus spp.) (e.g., Ferrari et al., 1989; Saunders et al., 1984; Hoch et al., 1967; Mann et al., 1986; Holubova, 1985; Chang et al., 1979; Vorobjeva et al., 1980; Smith et al., 1986; Fisher et. al., 1981 and McDonald, 1984). Indeed, such methods as transformation including protoplast transformation and congression, transduction, and protoplast fusion are known and suited for use in the present disclosure. Methods of transformation are particularly preferred to introduce a DNA construct of the present disclosure into a host cell.

In addition to commonly used methods, in some embodiments, Bacillus host cells are directly transformed (i.e., an intermediate cell is not used to amplify, or otherwise process, the DNA construct prior to introduction into the host cell). Introduction of the DNA construct into the host cell includes those physical and chemical methods known in the art to introduce DNA into a host cell, without insertion into a plasmid or vector. Such methods include, but are not limited to, calcium chloride precipitation, electroporation, naked DNA, liposomes and the like. In additional embodiments, DNA constructs are co-transformed with a plasmid without being inserted into the plasmid. In further embodiments, a selective marker is deleted or substantially excised from the modified Bacillus strain by methods known in the art (e.g., Stahl et al., 1984 and Palmeros et al., 2000). In some embodiments, resolution of the vector from a host chromosome leaves the flanking regions in the chromosome, while removing the indigenous chromosomal region.

Promoters and promoter sequence regions for use in the expression of genes, open reading frames (ORFs) thereof and/or variant sequences thereof in Bacillus cells are generally known on one of skill in the art. Promoter sequences of the disclosure of the disclosure are generally chosen so that they are functional in the Bacillus cells (e.g., B. licheniformis cells). Promoters useful for driving gene expression in Bacillus cells include, but are not limited to, the B. subtilis alkaline protease (aprE) promoter (Stahl et al., 1984), the a-amylase promoter of B. subtilis (Yang et al., 1983), the α-amylase promoter of B. amyloliquefaciens (Tarkinen et al., 1983), the neutral protease (nprE) promoter from B. subtilis (Yang et al., 1984), a mutant aprE promoter (PCT Publication No. WO2001/51643) or any other promoter from B. subtilis, B. licheniformis or other related Bacilli. Methods for screening and creating promoter libraries with a range of activities (promoter strength) in Bacillus cells is described in PCT Publication No. WO2003/089604.

### IV. Culturing Bacillus Cells for Production of a Protein of Interest

In certain embodiments the disclosure provides methods and compositions for increasing the protein productivity of a modified Bacillus cell, as compared (i.e., relative, vis-à-vis) to an unmodified (parental) cell. Thus, in certain embodiments the disclosure provides methods of producing a protein of interest (POI) comprising fermenting/cultivating a modified Bacillus cell, wherein the modified cell secrets the POI into the culture medium or retains the POI intracellularly. Fermentation methods well known in the art can be applied to ferment the modified and unmodified Bacillus cells of the disclosure.

For example, in some embodiments, the cells are cultured under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s). In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within typical batch cultures, cells can progress through a static lag phase to a high growth log phase, and finally to a stationary phase, where growth rate is diminished or halted. If untreated, cells in the stationary phase eventually die. In general, cells in log phase are responsible for the bulk of production of product.

A suitable variation on the standard batch system is the “fed-batch fermentation” system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO₂. Batch and fed-batch fermentations are common and known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

Thus, in certain embodiments, a POI produced by a transformed (modified) host cell may be recovered from the culture medium by conventional procedures including separating the host cells from the medium by centrifugation or filtration, or if necessary, disrupting the cells and removing the supernatant from the cellular fraction and debris. Typically, after clarification, the proteinaceous components of the supernatant or filtrate are precipitated by means of a salt, e.g., ammonium sulfate. The precipitated proteins are then solubilized and may be purified by a variety of chromatographic procedures, e.g., ion exchange chromatography, gel filtration.

### V. Proteins of Interest Produced by Modified (Host) Cells

A protein of interest (POI) of the instant disclosure can be any endogenous or heterologous protein, and it may be a variant of such a POI. The protein can contain one or more disulfide bridges or is a protein whose functional form is a monomer or a multimer, i.e., the protein has a quaternary structure and is composed of a plurality of identical (homologous) or non-identical (heterologous) subunits, wherein the POI or a variant POI thereof is preferably one with properties of interest.

Thus, in certain embodiments, a modified cell of the disclosure expresses an endogenous POI, a heterologous POI or a combination of one or more thereof.

In certain embodiments, a modified Bacillus cell of the disclosure exhibits an increased specific productivity (Qp) of a POI relative the (unmodified) parental Bacillus cell. For example, the detection of specific productivity (Qp) is a suitable method for evaluating protein production. The specific productivity (Qp) can be determined using the following equation:

“Qp=gP/gDCW·hr”

wherein, “gP” is grams of protein produced in the tank; “gDCW” is grams of dry cell weight (DCW) in the tank and “hr” is fermentation time in hours from the time of inoculation, which includes the time of production as well as growth time.

Thus, in certain other embodiments, a modified Bacillus cell of the disclosure comprises a specific productivity (Qp) increase of at least about 1%, at least about 5%, at least about 6%, at least about 7%, at least about 8%, at least about 9%, or at least about 10% or more as compared to the unmodified (parental) cell.

In certain embodiments, a POI or a variant POI thereof is selected from the group consisting of acetyl esterases, aryl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.

Thus, in certain embodiments, a POI or a variant POI thereof is an enzyme selected from Enzyme Commission (EC) Number EC 1, EC 2, EC 3, EC 4, EC 5 or EC 6.

For example, in certain embodiments a POI is an oxidoreductase enzyme, including, but not limited to, an EC 1 (oxidoreductase) enzyme selected from EC 1.10.3.2 (e.g., a laccase), EC 1.10.3.3 (e.g., L-ascorbate oxidase), EC 1.1.1.1 (e.g., alcohol dehydrogenase), EC 1.11.1.10 (e.g., chloride peroxidase), EC 1.11.1.17 (e.g., peroxidase), EC 1.1.1.27 (e.g., L-lactate dehydrogenase), EC 1.1.1.47 (e.g., glucose 1-dehydrogenase), EC 1.1.3.X (e.g., glucose oxidase), EC 1.1.3.10 (e.g., pyranose oxidase), EC 1.13.11.X (e.g., dioxygenase), EC 1.13.11.12 (e.g., lineolate 13S-lipozygenase), EC 1.1.3.13 (e.g., alcohol oxidase), EC 1.14.14.1 (e.g., monooxygenase), EC 1.14.18.1 (e.g., monophenol monooxigenase) EC 1.15.1.1 (e.g., superoxide dismutase), EC 1.1.5.9 (formerly EC 1.1.99.10, e.g., glucose dehydrogenase), EC 1.1.99.18 (e.g., cellobiose dehydrogenase), EC 1.1.99.29 (e.g., pyranose dehydrogenase), EC 1.2.1.X (e.g., fatty acid reductase), EC 1.2.1.10 (e.g., acetaldehyde dehydrogenase), EC 1.5.3.X (e.g., fructosyl amine reductase), EC 1.8.1.X (e.g., disulfide reductase) and EC 1.8.3.2 (e.g., thiol oxidase).

In certain embodiments a POI is a transferase enzyme, including, but not limited to, an EC 2 (transferase) enzyme selected from EC 2.3.2.13 (e.g., transglutaminase), EC 2.4.1.X (e.g., hexosyltransferase), EC 2.4.1.40 (e.g., alternasucrase), EC 2.4.1.18 (e.g., 1,4 alpha-glucan branching enzyme), EC 2.4.1.19 (e.g., cyclomaltodextrin glucanotransferase), EC 2.4.1.2 (e.g., dextrin dextranase), EC 2.4.1.20 (e.g., cellobiose phosphorylase), EC 2.4.1.25 (e.g., 4-alpha-glucanotransferase), EC 2.4.1.333 (e.g., 1,2-beta-oligoglucan phosphor transferase), EC 2.4.1.4 (e.g., amylosucrase), EC 2.4.1.5 (e.g., dextransucrase), EC 2.4.1.69 (e.g., galactoside 2-alpha-L-fucosyl transferase), EC 2.4.1.9 (e.g., inulosucrase), EC 2.7.1.17 (e.g., xylulokinase), EC 2.7.7.89 (formerly EC 3.1.4.15, e.g., [glutamine synthetase]-adenylyl-L-tyrosine phosphorylase), EC 2.7.9.4 (e.g., alpha glucan kinase) and EC 2.7.9.5 (e.g., phosphoglucan kinase).

In other embodiments a POI is a hydrolase enzyme, including, but not limited to, an EC 3 (hydrolase) enzyme selected from EC 3.1.X.X (e.g., an esterase), EC 3.1.1.1 (e.g., pectinase), EC 3.1.1.14 (e.g., chlorophyllase), EC 3.1.1.20 (e.g., tannase), EC 3.1.1.23 (e.g., glycerol-ester acylhydrolase), EC 3.1.1.26 (e.g., galactolipase), EC 3.1.1.32 (e.g., phospholipase Al), EC 3.1.1.4 (e.g., phospholipase A2), EC 3.1.1.6 (e.g., acetylesterase), EC 3.1.1.72 (e.g., acetylxylan esterase), EC 3.1.1.73 (e.g., feruloyl esterase), EC 3.1.1.74 (e.g., cutinase), EC 3.1.1.86 (e.g., rhamnogalacturonan acetylesterase), EC 3.1.1.87 (e.g., fumosin B1 esterase), EC 3.1.26.5 (e.g., ribonuclease P), EC 3.1.3.X (e.g., phosphoric monoester hydrolase), EC 3.1.30.1 (e.g., Aspergillus nuclease S1), EC 3.1.30.2 (e.g., Serratia marcescens nuclease), EC 3.1.3.1 (e.g., alkaline phosphatase), EC 3.1.3.2 (e.g., acid phosphatase), EC 3.1.3.8 (e.g., 3-phytase), EC 3.1.4.1 (e.g., phosphodiesterase I), EC 3.1.4.11 (e.g., phosphoinositide phospholipase C), EC 3.1.4.3 (e.g., phospholipase C), EC 3.1.4.4 (e.g., phospholipase D), EC 3.1.6.1 (e.g., arylsufatase), EC 3.1.8.2 (e.g., diisopropyl-fluorophosphatase), EC 3.2.1.10 (e.g., oligo-1,6-glucosidase), EC 3.2.1.101 (e.g., mannan endo-1,6-alpha-mannosidase), EC 3.2.1.11 (e.g., alpha-1,6-glucan-6-glucanohydrolase), EC 3.2.1.131 (e.g., xylan alpha-1,2-glucuronosidase), EC 3.2.1.132 (e.g., chitosan N-acetylglucosaminohydrolase), EC 3.2.1.139 (e.g., alpha-glucuronidase), EC 3.2.1.14 (e.g., chitinase), EC 3.2.1.151 (e.g., xyloglucan-specific endo-beta-1,4-glucanase), EC 3.2.1.155 (e.g., xyloglucan-specific exo-beta-1,4-glucanase), EC 3.2.1.164 (e.g., galactan endo-1,6-beta-galactosidase), EC 3.2.1.17 (e.g., lysozyme), EC 3.2.1.171 (e.g., rhamnogalacturonan hydrolase), EC 3.2.1.174 (e.g., rhamnogalacturonan rhamnohydrolase), EC 3.2.1.2 (e.g., beta-amylase), EC 3.2.1.20 (e.g., alpha-glucosidase), EC 3.2.1.22 (e.g., alpha-galactosidase), EC 3.2.1.25 (e.g., beta-mannosidase), EC 3.2.1.26 (e.g., beta-fructofuranosidase), EC 3.2.1.37 (e.g., xylan 1,4-beta-xylosidase), EC 3.2.1.39 (e.g., glucan endo-1,3-beta-D-glucosidase), EC 3.2.1.40 (e.g., alpha-L-rhamnosidase), EC 3.2.1.51 (e.g., alpha-L-fucosidase), EC 3.2.1.52 (e.g., beta-N-Acetylhexosaminidase), EC 3.2.1.55 (e.g., alpha-N-arabinofuranosidase), EC 3.2.1.58 (e.g., glucan 1,3-beta-glucosidase), EC 3.2.1.59 (e.g., glucan endo-1,3-alpha-glucosidase), EC 3.2.1.67 (e.g., galacturan 1,4-alpha-galacturonidase), EC 3.2.1.68 (e.g., isoamylase), EC 3.2.1.7 (e.g., 1-beta-D-fructan fructanohydrolase), EC 3.2.1.74 (e.g., glucan 1,4-β-glucosidase), EC 3.2.1.75 (e.g., glucan endo-1,6-beta-glucosidase), EC 3.2.1.77 (e.g., mannan 1,2-(1,3)-alpha-mannosidase), EC 3.2.1.80 (e.g., fructan beta-fructosidase), EC 3.2.1.82 (e.g., exo-poly-alpha-galacturonosidase), EC 3.2.1.83 (e.g., kappa-carrageenase), EC 3.2.1.89 (e.g., arabinogalactan endo-1,4-beta-galactosidase), EC 3.2.1.91 (e.g., cellulose 1,4-beta-cellobiosidase), EC 3.2.1.96 (e.g., mannosyl-glycoprotein endo-beta-N-acetylglucosaminidase), EC 3.2.1.99 (e.g., arabinan endo-1,5-alpha-L-arabinanase), EC 3.4.X.X (e.g., peptidase), EC 3.4.11.X (e.g., aminopeptidase), EC 3.4.11.1 (e.g., leucyl aminopeptidase), EC 3.4.11.18 (e.g., methionyl aminopeptidase), EC 3.4.13.9 (e.g., Xaa-Pro dipeptidase), EC 3.4.14.5 (e.g., dipeptidyl-peptidase IV), EC 3.4.16.X (e.g., serine-type carboxypeptidase), EC 3.4.16.5 (e.g., carboxypeptidase C), EC 3.4.19.3 (e.g., pyroglutamyl-peptidase I), EC 3.4.21.X (e.g., serine endopeptidase), EC 3.4.21.1 (e.g., chymotrypsin), EC 3.4.21.19 (e.g., glutamyl endopeptidase), EC 3.4.21.26 (e.g., prolyl oligopeptidase), EC 3.4.21.4 (e.g., trypsin), EC 3.4.21.5 (e.g., thrombin), EC 3.4.21.63 (e.g., oryzin), EC 3.4.21.65 (e.g., thermomycolin), EC 3.4.21.80 (e.g., streptogrisin A), EC 3.4.22.X (e.g., cysteine endopeptidase), EC 3.4.22.14 (e.g., actinidain), EC 3.4.22.2 (e.g., papain), EC 3.4.22.3 (e.g., ficain), EC 3.4.22.32 (e.g., stem bromelain), EC 3.4.22.33 (e.g., fruit bromelain), EC 3.4.22.6 (e.g., chymopapain), EC 3.4.23.1 (e.g., pepsin A), EC 3.4.23.2 (e.g., pepsin B), EC 3.4.23.22 (e.g., endothiapepsin), EC 3.4.23.23 (e.g., mucorpepsin), EC 3.4.23.3 (e.g., gastricsin), EC 3.4.24.X (e.g., metalloendopeptidase), EC 3.4.24.39 (e.g., deuterolysin), EC 3.4.24.40 (e.g., serralysin), EC 3.5.1.1 (e.g., asparaginase), EC 3.5.1.11 (e.g., penicillin amidase), EC 3.5.1.14 (e.g., N-acyl-aliphatic-L-amino acid amidohydrolase), EC 3.5.1.2 (e.g., L-glutamine amidohydrolase), EC 3.5.1.28 (e.g., N-acetylmuramoyl-L-alanine amidase), EC 3.5.1.4 (e.g., amidase), EC 3.5.1.44 (e.g., protein-L-glutamine amidohydrolase), EC 3.5.1.5 (e.g., urease), EC 3.5.1.52 (e.g., peptide-N(4)-(N-acetyl-beta-glucosaminyl)asparagine amidase), EC 3.5.1.81 (e.g., N-Acyl-D-amino-acid deacylase), EC 3.5.4.6 (e.g., AMP deaminase) and EC 3.5.5.1 (e.g., nitrilase).

In other embodiments a POI is a lyase enzyme, including, but not limited to, an EC 4 (lyase) enzyme selected from EC 4.1.2.10 (e.g., mandelonitrile lyase), EC 4.1.3.3 (e.g., N-acetylneuraminate lyase), EC 4.2.1.1 (e.g., carbonate dehydratase), EC 4.2.2.- (e.g., rhamnogalacturonan lyase), EC 4.2.2.10 (e.g., pectin lyase), EC 4.2.2.22 (e.g., pectate trisaccharide-lyase), EC 4.2.2.23 (e.g., rhamnogalacturonan endolyase) and EC 4.2.2.3 (e.g., mannuronate-specific alginate lyase).

In certain other embodiments a POI is an isomerase enzyme, including, but not limited to, an EC 5 (isomerase) enzyme selected from EC 5.1.3.3 (e.g., aldose 1-epimerase), EC 5.1.3.30 (e.g., D-psicose 3-epimerase), EC 5.4.99.11 (e.g., isomaltulose synthase) and EC 5.4.99.15 (e.g., (1→4)-α-D-glucan 1-α-D-glucosylmutase).

In yet other embodiments, a POI is a ligase enzyme, including, but not limited to, an EC 6 (ligase) enzyme selected from EC 6.2.1.12 (e.g., 4-coumarate:coenzyme A ligase) and EC 6.3.2.28 (e.g., L-amino-acid alpha-ligase).

Thus, in certain embodiments, industrial protease producing Bacillus host cells provide particularly preferred expression hosts. Likewise, in certain other embodiments, industrial amylase producing Bacillus host cells provide particularly preferred expression hosts.

For example, there are two general types of proteases which are typically secreted by Bacillus spp., namely neutral (or “metalloproteases”) and alkaline (or “serine”) proteases. For example, Bacillus subtilisin proteins (enzymes) are exemplary serine proteases for use in the present disclosure. A wide variety of Bacillus subtilisins have been identified and sequenced, for example, subtilisin 168, subtilisin BPN′, subtilisin Carlsberg, subtilisin DY, subtilisin 147 and subtilisin 309 (e.g., WO 1989/06279 and Stahl et al., 1984). In some embodiments of the present disclosure, the modified Bacillus cells produce mutant (i.e., variant) proteases. Numerous references provide examples of variant proteases, such as PCT Publication Nos. WO1999/20770; WO1999/20726; WO1999/20769; WO1989/06279; U.S. RE34,606; U.S. Pat. Nos. 4,914,031; 4,980,288; 5,208,158; 5,310,675; 5,336,611; 5,399,283; 5,441,882; 5,482,849; 5,631,217; 5,665,587; 5,700,676; 5,741 ,694; 5,858,757; 5,880,080; 6,197,567 and 6,218,165. Thus, in certain embodiments, a modified Bacillus cells of the disclosure comprises an expression construct encoding a protease.

In certain other embodiments, a modified Bacillus cells of the disclosure comprises an expression construct encoding an amylase. A wide variety of amylase enzymes and variants thereof are known to one skilled in the art. For example, International PCT Publication NO. WO2006/037484 and WO 2006/037483 describe variant α-amylases having improved solvent stability, Publication No. WO1994/18314 discloses oxidatively stable α-amylase variants, Publication No. WO1999/19467, WO2000/29560 and WO2000/60059 disclose Termamyl-like α-amylase variants, Publication No. WO2008/112459 discloses α-amylase variants derived from Bacillus sp. number 707, Publication No. WO1999/43794 discloses maltogenic α-amylase variants, Publication No. WO1990/11352 discloses hyper-thermostable α-amylase variants, Publication No. WO2006/089107 discloses α-amylase variants having granular starch hydrolyzing activity.

In other embodiments, a POI or variant POI expressed and produced in a modified cell of the disclosure is a peptide, a peptide hormone, a growth factor, a clotting factor, a chemokine, a cytokine, a lymphokine, an antibody, a receptor, an adhesion molecule, a microbial antigen (e.g., HBV surface antigen, HPV E7, etc.), variants thereof, fragments thereof and the like. Other types of proteins (or variants thereof) of interest may be those that are capable of providing nutritional value to a food or to a crop. Non-limiting examples include plant proteins that can inhibit the formation of anti-nutritive factors and plant proteins that have a more desirable amino acid composition (e.g., a higher lysine content than a non-transgenic plant).

There are various assays known to those of ordinary skill in the art for detecting and measuring activity of intracellularly and extracellularly expressed proteins. In particular, for proteases, there are assays based on the release of acid-soluble peptides from casein or hemoglobin measured as absorbance at 280 nm or colorimetrically, using the Folin method (e.g., Bergmeyer et al., 1984). Other assays involve the solubilization of chromogenic substrates (See e.g., Ward, 1983). Other exemplary assays include succinyl-Ala-Ala-Pro-Phe-para-nitroanilide assay (SAAPFpNA) and the 2,4,6-trinitrobenzene sulfonate sodium salt assay (TNBS assay). Numerous additional references known to those in the art provide suitable methods (See e.g., Wells et al., 1983; Christianson et al., 1994 and Hsia et al., 1999).

International PCT Publication No. WO2014/164777 discloses Ceralpha α-amylase activity assays useful for amylase activities described herein.

Means for determining the levels of secretion of a protein of interest in a host cell and detecting expressed proteins include the use of immunoassays with either polyclonal or monoclonal antibodies specific for the protein. Examples include enzyme-linked immunosorbent assay (ELISA), radioimmunoassay (RIA), fluorescence immunoassay (FIA), and fluorescent activated cell sorting (FACS).

### EXAMPLES

Certain aspects of the present invention may be further understood in light of the following examples, which should not be construed as limiting. Modifications to materials and methods will be apparent to those skilled in the art.

### Example 1

### Construction of—1A UTR Expression Constructs and Testing of Amylase Production

The effect of modified 5′ untranslated regions (5′-UTRs) on expression of genes encoding proteins of interest in Bacillus cells was tested by creating expression cassettes of either the wild-type B. subtilis aprE 5′ UTR (SEQ ID NO: 1) or a modified 5′ UTR (SEQ ID NO: 2) (e.g., see FIG. 1). More particularly, the instant example describes the creation of Bacillus strains/host cells for the assessment of various (modified) 5′ UTR constructs, and their impact/influence on the production of proteins of interest when such modified 5′ UTR constructs are operably linked to an upstream (5′) promoter and a downstream (3′) open reading frame encoding the protein of interest.

In the present example, parental B. licheniformis cells comprising a plasmid carrying a xylose-inducible comK coding sequence (SEQ ID NO: 3), were grown overnight at 37° C. and 250 RPM in 15 ml of L broth (1% (w/v) Tryptone, 0.5% Yeast extract (w/v), 1% NaCl (w/v)) containing 100 μg/ml spectinomycin dihydrochloride in a 125 ml baffled flask. The overnight culture was diluted to 0.7 OD₆₀₀ units in 25 ml fresh L broth containing 100 μg/ml spectinomycin dihydrochloride in a 250 ml baffle flask. Cells were grown for 1 hour at 37° C. (250 RPM). D-xylose was added to 0.1% (w/v) from a 50% (w/v) stock. Cells were grown for an additional 4 hours at 37° C. (250 RPM) and pelleted at 1700×g for 7 minutes.

The cells were resuspended in ¼ volume of original culture using the spent medium. One hundred (100) μl of concentrated cells were mixed with approximately 1 μg of either the wild-type (WT) 5′ UTR expression construct (WT-5′ UTR; SEQ ID NO: 4) or the modified 5′ UTR expression construct (mod-5′ UTR; SEQ ID NO: 5). For example, each expression cassette comprised (in the 5′ to 3′ direction) the same 5′ catH homology arm (SEQ ID NO: 6), catH gene (SEQ ID NO: 7) and spoVGrrnlp hybrid promoter (SEQ ID NO: 8), operably linked to either the wild-type B. subtilis aprE 5′ UTR (SEQ ID NO: 1) or the modified aprE 5′ UTR (SEQ ID NO: 2). In addition, the 5′ UTR was operably linked to the DNA encoding the amylase signal sequence of SEQ ID NO: 9), followed by DNA (ORF) of SEQ ID NO: 10 encoding a variant G. stearothermophilus α-amylase of SEQ ID NO: 13 (e.g., see PCT Publication No. WO2009/134670, incorporated herein by reference in its entirety). The 3′ end of the DNA (ORF) encoding variant G. stearothermophilus α-amylase (SEQ ID NO: 10) was operably linked to the amylase terminator of SEQ ID NO: 11, which was operably linked to the 3′ catH homology arm (SEQ ID NO: 12). Transformation reactions were incubated at 37° C., 1000 RPM for approximately 90 minutes.

Transformation mixes were plated on petri plates filled with L-broth containing 10 μg/ml chloramphenicol solidified with 1.5% (w/v) agar. Plates were incubated at 37° C. for 2 days. Colonies were streak purified on petri plates filled with L-broth containing 1% (w/v) insoluble corn starch solidified with 1.5% (w/v) agar. Plates were incubated at 37° C. for 24 hours until colonies had formed. Starch hydrolysis was indicated by clearing of the insoluble starch surrounding the colony, forming a halo, and was used to select transformants expressing variant G. stearothermophilus α-amylase protein (SEQ ID NO: 13). Colony PCR was used to amplify the catH locus (WT construct; SEQ ID NO: 14) (modified construct, SEQ ID NO: 15) from halo producing colonies using standard techniques, and primer pairs: forward primer (TGTGTGACGGCTATCATGCC; SEQ ID NO: 16)/reverse primer (TTGAGAGCCGGCGTTCC; SEQ ID NO: 17). PCR products were purified from excess primers and nucleotides using standard techniques and sequenced using the method of Sanger and the following sequencing primers:

(SEQ ID NO: 18)
AACGAGTTGGAACGGCTTGC; forward,
(SEQ ID NO: 19)
GGCAACACCTACTCCAGCTT; forward,
(SEQ ID NO: 20)
GATCACTCCGACATCATCGG; forward.

A sequence verified B. licheniformis (daughter) cell comprising the WT-5′ UTR expression cassette (SEQ ID NO: 4) was stored as daughter cell BF134, and a sequence verified B. licheniformis (daughter) cell comprising the modified 5′-UTR (mod-5′ UTR) expression cassette (SEQ ID NO: 5) was stored as daughter cell BF117.

### Example 2

### Assessment of the Effect Modified 5′ UTRS Have of the Production of a Protein of Interest

In the instant example, the modified B. licheniformis daughter cells described above in Example 1 (i.e., daughter cells BF134; comprising SEQ ID NO: 4 and daughter cells BF117, comprising (SEQ ID NO: 5) were grown under standard fermentation conditions for 84 hours. More specifically, the relative amylase production of the B. licheniformis daughter cells BF134 and BF117 was measured using standard methods, the results of which are set forth below in Table 1.

As presented in Table 1, the BF117 cells (comprising the mod-5′ UTR expression construct; SEQ ID NO: 5) produced 20% more amylase than the BF134 cells (comprising the WT-5′ UTR expression construct; SEQ ID NO: 4). More particularly, an a per OD₅₅₀ unit basis, the BF117 cells produced 40% more amylase than the BF134 cells.

TABLE 1
AMYLASE PRODUCTIVITY OF MODIFIED B. LICHENIFORMIS CELLS

| | | Relative | | |
|---|---|---|---|---|
| B. licheniformis | | Amylase | Relative | Relative |
| Daughter Cell | Expression Construct | Expression | OD₆₀₀ | Amylase/OD₆₀₀ |
| BF134 | WT-5′ UTR (SEQ ID NO: 4) | 1.0 | 1.00 | 1.0 |
| BF117 | mod-5′UTR (SEQ ID NO: 5) | 1.2 | 0.84 | 1.4 |

### REFERENCES

PCT International Publication No. WO1989/06279
PCT International Publication No. WO1999/20726
PCT International Publication No. WO1999/20769
PCT International Publication No. WO1999/20770
PCT International Publication No. WO2002/14490
PCT International Publication No. WO2003/083125
PCT International Publication No. WO2003/089604
PCT International Publication No. WO2014/164777
U.S. Pat No. 4,914,031
U.S. Pat No. 4,980,288
U.S. Pat No. 5,208,158
U.S. Pat No. 5,310,675
U.S. Pat No. 5,336,611
U.S. Pat No. 5,399,283
U.S. Pat No. 5,441,882
U.S. Pat No. 5,482,849
U.S. Pat No. 5,631,217
U.S. Pat No. 5,665,587
U.S. Pat No. 5,700,676
U.S. Pat No. 5,741,694
U.S. Pat No. 5,858,757
U.S. Pat No. 5,880,080
U.S. Pat No. 6,197,567
U.S. Pat No. 6,218,165
U.S. Patent Publication No. 2014/0329309
U.S. RE 34,606

Bansal, “Relative stability of DNA as a generic criterion for promoter prediction: whole genome annotation of microbial genomes with varying nucleotide base composition”, Mol. Biosyst., 5: 1758-1769, 2009.
Bergmeyer et al., “Methods of Enzymatic Analysis” vol. 5, Peptidases, Proteinases and their Inhibitors, Verlag Chemie, Weinheim, 1984.
Botstein and Shortie, Science 229: 4719, 1985.
Brode et al., “Subtilisin BPN′ variants: increased hydrolytic activity on surface-bound substrates via decreased surface activity”, Biochemistry, 35(10):3162-3169, 1996.
Caspers et al., “Improvement of Sec-dependent secretion of a heterologous model protein in Bacillus subtilis by saturation mutagenesis of the N-domain of the AmyE signal peptide”, Appl. Microbiol. Biotechnol., 86(6):1877-1885, 2010.
Chang et al., Mol. Gen. Genet., 168:11-115, 1979.
Christianson et al., Anal. Biochem., 223:119 -129, 1994.
de Avila et al., “BacPP: bacterial promoter prediction—a tool for accurate sigma-factor specific assignment in enterobacteria”, J. Theor. Biol., 287, 92-99, 2011.
Devereux et at, Nucl. Acid Res., 12: 387-395, 1984.
Dontsova et al., “The location of mRNA in the ribosomal 30S initiation complex; site-directed cross-linking of mRNA analogues carrying several photoreactive labels simultaneously on either side of the AUG start codon”, EMBO J., 10:2613-2620, 1991.
Earl et al., “Ecology and genomics of Bacillus subtilis”, Trends in Microbiology.,16(6): 269-275, 2008.
Ferrari et al., “Genetics,” in Harwood et al. (ed), Bacillus, Plenum Publishing Corp., 1989.
Fisher et. al., Arch. Microbiol., 139:213-217, 1981.
Haldenwang et al., Microbiol Reviews 59(1):1-30, 1995.
Hamoen et al., Genes Dev. 12:1539- 1550, 1998.
Higuchi et al., Nucleic Acids Research 16: 7351, 1988.
Ho et al., Gene 77: 61, 1989.
Hoch et al., J. Bacteriol., 93:1925 -1937, 1967.
Holubova, Folia Microbiol., 30:97, 1985.
Hopwood, The Isolation of Mutants in Methods in Microbiology (J. R. Norris and D. W. Ribbons, eds.) pp 363-433, Academic Press, New York, 1970.
Horton et al., Gene 77: 61, 1989.
Hsia et al., Anal Biochem., 242:221-227, 1999.
Iglesias and Trautner, Molecular General Genetics 189: 73-76, 1983.
Jacob et al., “A single base change in the Shine-Dalgarno region of the 16S rRNA of E. coli affects translation of many proteins”, PNAS, 84:4757-4761, 1987.
Jacques and Dreyfus, “Translation initiation in E. coli: old and new questions”, Mol. Microbiol. 4: 1063-1067, 1990.
Jensen et al., “Cell-associated degradation affects the yield of secreted engineered and heterologous proteins in the Bacillus subtilis expression system” Microbiology, 146 (Pt 10:2583-2594, 2000.
Klucar et al., Nucleic Acids Res. 38:D366-D370, 2010.
Liu and Zuber, 1998,
Lo et al., Proceedings of the National Academy of Sciences USA 81: 2285, 1985.
Mann et al., Current Microbiol., 13:131 -135, 1986.
McDonald, J. Gen. Microbiol., 130:203, 1984.
Munch et al., Nucleic Acids Res., 31(1):266-269, 2003
Needleman and Wunsch, J. Mol. Biol., 48: 443, 1970.
Olempska-Beer et al., “Food-processing enzymes from recombinant microorganisms--a review”' Regul. Toxicol. Pharmacol., 45(2):144-158, 2006.
Palmeros et al., Gene 247:255 -264, 2000.
Parish and Stoker, FEMS Microbiology Letters 154: 151-157, 1997.
Pearson and Lipman, Proc. Natl. Acad. Sci. USA 85: 2444, 1988.
Perego, 1993, In A. L. Sonneshein, J. A. Hoch, and R. Losick, editors, Bacillus subtilis and Other Gram-Positive Bacteria, Chapter 42, American Society ofMicrobiology, Washington, D.C.
Raul et al., “Production and partial purification of alpha amylase from Bacillus subtilis (MTCC 121) using solid state fermentation”, Biochemistry Research International, 2014.
Salamov, “Automatic annotation of microbial genomes and metagenomic sequences”, In: Li RW (ed), Metagenomics and its applications in agriculture, biomedicine and environmental studies. Nova Science Publishers, Hauppauge, N.Y., pp. 61-78, 2011.
Sarkar and Sommer, BioTechniques 8: 404, 1990.
Saunders et al., J. Bacteriol., 157: 718-726, 1984.
Shimada, Meth. Mol. Biol. 57: 157; 1996
Shine and Dalgarno, “Determinant of cistron specificity in bacterial ribosomes”, Nature, 254: 34-38, 1975.
Shine and Dalgarno, “The 3′-terminal sequence of E. coli 16s ribosomal RNA: complementary to nonsense triplets and ribosome binding sites”, PNAS, 71: 1342-1346, 1974.
Smith and Waterman, Adv. Appl. Math., 2: 482, 1981.
Smith et al., Appl. Env. Microbiol., 51 :634 1986.
Stahl and Ferrari, J. Bacteriol., 158:411-418, 1984.
Stahl et al, J. Bacteriol., 158 :411-418, 1984.
Tarkinen, et al, J. Biol. Chem. 258: 1007-1013, 1983.
Van Dijl and Hecker, “Bacillus subtilis: from soil bacterium to super-secreting cell factory”, Microbial Cell Factories, 12(3). 2013.
Vorobjeva et al., FEMS Microbiol. Lett., 7:261-263, 1980.
Ward, “Proteinases,” in Fogarty (ed)., Microbial Enzymes and Biotechnology. Applied Science, London, pp 251-317, 1983.
Wells et al., Nucleic Acids Res. 11 :7911-7925, 1983.
Westers et al., “Bacillus subtilis as cell factory for pharmaceutical proteins: a biotechnological approach to optimize the host organism”, Biochimica et Biophysica Acta., 1694:299-310, 2004.
Yang et al, J. Bacteriol.,160: 15-21, 1984.
Yang et al., Nucleic Acids Res. 11: 237-249, 1983.
Youngman et al., Proc. Natl. Acad. Sci. USA 80: 2305-2309, 1983.

1. An isolated polynucleotide comprising a modified Bacillus subtilis aprE 5′-untranslated region (mod-5′-UTR) nucleic acid sequence derived from a wild-type Bacillus subtilis aprE 5′-untranslated region (WT-5′-UTR) nucleic acid sequence SEQ ID NO: 1.

2. The polynucleotide of claim 1, wherein the mod-5′-UTR comprises SEQ ID NO: 2.

3. The polynucleotide of claim 1, wherein the mod-5′-UTR further comprises an upstream (5′) promoter region nucleic acid sequence 5′ and operably linked to the mod-5′-UTR.

4. The polynucleotide of claim 1, wherein the mod-5′-UTR further comprises a downstream (3′) open reading frame (ORF) nucleic acid sequence encoding a protein of interest, wherein the ORF sequence is 3′ and operably linked to the mod-5′-UTR.

5. The polynucleotide of claim 1, comprising Formula (I) in the 5′ to 3′ direction:
[Pro][mod-5′-UTR][ORF]; (I):
wherein [Pro] is a promoter region nucleic acid sequence operable in a Bacillus sp. cell, [mod-5′-UTR] is a modified B. subtilis aprE 5′ untranslated region (mod-5′-UTR) nucleic acid sequence and [ORF] is an open reading frame nucleic acid sequence encoding a protein of interest (POI), wherein the [Pro], [mod-5′-UTR] and [ORF] nucleic acid sequences are operably linked.

6. A vector comprising the polynucleotide of claim 1.

7. A DNA expression construct comprising the polynucleotide of claim 1.

8. A Bacillus sp. cell comprising the polynucleotide of claim 1.

9. The Bacillus sp. cell of claim 8, wherein the cell is a Bacillus licheniformis cell.

10. An isolated polynucleotide comprising a modified Bacillus sp. 5′-UTR (mod-5′-UTR) nucleic acid sequence derived from a wild-type Bacillus sp. 5′-UTR (WT-5′-UTR) sequence, the isolated modified polynucleotide comprising in the 5′ to 3′ direction the nucleic acid sequences of Formula (II) in operable combination:
[TIS][mod-5′ UTR][tss codon] (II):
wherein [TIS] is transcription initiation site (TIS), [mod-5′-UTR] comprises a modified B. subtilis 5′-UTR nucleic acid sequence and [tss codon] is a three (3) nucleotide translation start site (tss) codon

11. The polynucleotide of claim 10, wherein the [mod-5′-UTR] sequence comprises SEQ ID NO: 2.

12. The polynucleotide of claim 10, further comprising:
(a) a nucleic acid promoter sequence upstream (5′) and operably linked to the [TIS], which promoter sequence is operable in a Bacillus sp. cell and
(b) an ORF nucleic acid sequence downstream (3′) and operably linked to the tss codon, wherein the ORF sequence encodes a POI.

13. A vector comprising the polynucleotide of claim 10.

14. A DNA expression construct comprising the polynucleotide of claim 10.

15. A Bacillus sp. cell comprising the polynucleotide of claim 10.

16. The Bacillus sp. cell of claim 15, wherein the cell is a Bacillus licheniformis cell.

17. An isolated polynucleotide comprising nucleic acid sequences of Formula (III) in the 5′ to 3′ direction and in operable combination,
[5′-HR][TIS ][mod-5′-UTR][tss codon][3′-HR], (III):
wherein [TIS] is the transcription initiaition site (TIS) , [mod-5′-UTR] comprises a modified B. subtilis 5′-UTR nucleic acid sequence, [tss codon] is a three (3) nucleotide translation start site (tss) codon, [5′-HR] is a 5′-nucleic acid sequence homology region and [3′-HR] is a 3′-nucleic acid sequence homology region, wherein the 5′-HR and 3′-HR comprise sufficient homology to a genomic (chromosomal) region (locus) immediately upstream (5′) of the [TIS ] sequence and immediately downstream (3′) of the [tss codon] sequence, respectively, to effect integration of the introduced polynucleotide construct into the genome of the modified Bacillus cell by homologous recombination.

18. A vector comprising the polynucleotide of claim 17.

19. A DNA expression construct comprising the polynucleotide of claim 17.

20. A Bacillus sp. cell comprising the polynucleotide of claim 17.

21. The Bacillus sp. cell of claim 17, wherein the cell is a Bacillus licheniformis cell.

22. The polynucleotide of claim 1, wherein the ORF sequence encodes a POI selected from the group consisting of acetyl esterases, aryl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.

23. The polynucleotide of claim 12, wherein the ORF sequence encodes a POI selected from the group consisting of acetyl esterases, aryl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.

24. An isolated polynucleotide comprising a modified-5′-UTR (mod-5′-UTR) nucleic acid sequence derived from a wild-type Bacillus sp. 5′-UTR (WT-5′-UTR) sequence, the isolated polynucleotide comprising in the 5′ to 3′ direction and operable combination the nucleic acid sequences of Formula (IV):
[TIS][5′-UTR ⁻ΔxN][tss codon], (IV):
wherein [TIS] is the transcription initiation site (TIS), [tss codon] is a three (3) nucleotide translation start site (tss) codon and [5′-UTR ⁻ΔxN] is a modified Bacillus sp. 5′-UTR nucleic acid sequence derived from a wild-type Bacillus sp. 5′-UTR nucleic acid sequence, wherein the mod-5′-UTR nucleic acid sequence [5′-UTR ⁻ΔxN] comprises a deletion (⁻Δ) of “x” nucleotides (“N”) at the distal (3′) end of the WT-5′-UTR nucleic acid sequence.

25. An isolated polynucleotide comprising a modified-5′-UTR (mod-5′-UTR) nucleic acid sequence derived from a wild-type Bacillus sp. 5′-UTR (WT-5′-UTR) sequence, the isolated polynucleotide comprising in the 5′ to 3′ direction and operable combination the nucleic acid sequences of Formula (V):
[TIS ][5′-UTR ⁺ΔxN][tss codon], (V):
wherein [TIS] is the transcription initiation site, [tss codon] is a three (3) nucleotide translation start site (tss) codon and [5′-UTR ΔxN] is a modified Bacillus sp. 5′-UTR nucleic acid sequence derived from a wild-type Bacillus sp. 5′-UTR nucleic acid sequence, wherein the mod-5′-UTR nucleic acid sequence [5′-UTR ⁺ΔxN] comprises an addition (⁺Δ) of “x” nucleotides (“N”) at the distal (3′) end of the wild-type Bacillus sp. 5′-UTR nucleic acid sequence.

26. A vector comprising the polynucleotide of claim 24 or 25.

27. A DNA expression construct comprising the polynucleotide of claim 24 or 25.

28. A Bacillus sp. cell comprising the polynucleotide of claim 24 or 25.

29. The Bacillus sp. cell of claim 24 or 25, wherein the cell is a Bacillus licheniformis cell.

30. A modified Bacillus sp. (daughter) cell producing an increased amount of a heterologous protein of interest (POI) when cultivated in a medium suitable for the production of a heterologous POI, the modified Bacillus cell comprising an introduced expression construct comprising nucleic acid sequences of Formula (I) in the 5′ to 3′ direction and in operable combination,
[Pro][mod-5′-UTR][ORF]; (I):
wherein [Pro] is a promoter region nucleic acid sequence operable in a Bacillus sp. cell, [mod-5′-UTR] is a modified B. subtilis untranslated region (mod-5′-UTR) nucleic acid sequence and [ORF] is an open reading frame nucleic acid sequence encoding a protein of interest (POI), wherein the [Pro], [mod-5′-UTR] and [ORF] nucleic acid sequences are operably linked. wherein the modified Bacillus (daughter) cell produces an increased amount of the heterologous POI relative to an unmodified B. licheniformis (parental) cell producing the same POI, when cultivated under similar conditions.

31. The Bacillus cell of claim 30, wherein the mod-5′-UTR comprises SEQ ID NO: 2.

32. The Bacillus cell of claim 30, wherein the cell is a Bacillus licheniformis cell.

33. The Bacillus cell of claim 30, wherein the ORF sequence encodes a POI selected from the group consisting of acetyl esterases, aryl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.

34. A method for producing an increased amount of a heterologous protein of interest (POI) in a modified Bacillus cell comprising:
(a) introducing into a parental Bacillus sp. cell an expression construct comprising in the 5′ to 3′ direction and in operable combination, nucleic acid sequences [Pro] [mod-5′-UTR] [ORF], wherein [Pro] is a promoter region nucleic acid sequence operable in a Bacillus sp. cell, [mod-5′-UTR] is a mod-5′-UTR nucleic acid sequence of SEQ ID NO: 2 and [ORF] is an open reading frame nucleic acid sequence encoding a protein of interest (POI), and
(b) cultivating the modified Bacillus sp. cell of step (a) in a medium suitable for the production of a heterologous POI,
wherein the modified Bacillus (daughter) cell produces an increased amount of the POI relative to a Bacillus control cell cultivated in the same medium of step (b), wherein the Bacillus control cell comprises an introduced expression construct comprising in the 5′ to 3′ direction and in operable combination nucleic acid sequences [Pro] [WT-5′-UTR] [ORF], wherein the [Pro] and [ORF] nucleic acid sequences are identical to the [Pro] and [ORF] sequence in step (a) and the [WT-5′-UTR] comprises SEQ ID NO: 1.

35. The Bacillus cell of claim 34, wherein the cell is a Bacillus licheniformis cell.

36. The Bacillus cell of claim 34, wherein the ORF sequence encodes a POI selected from the group consisting of acetyl esterases, aryl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof.

37. A method for producing an increased amount of an endogenous protein of interest (POI) in a modified Bacillus cell comprising:
(a) obtaining a parental Bacillus cell producing an endogenous POI,
(b) introducing into the cell of step (a) a polynucleotide construct comprising nucleic acid sequences of Formula (VI) in the 5′ to 3′ direction and in operable combination,
[5′-HR][mod-5′-UTR][3′-HR], (VI):
wherein [mod-5′-UTR] comprises SEQ ID NO: 2, [5′-HR] is a 5′-nucleic acid sequence homology region comprising homology to the genomic locus immediately upstream (5′) of the endogenous wild-type 5′-UTR (WT-5′-UTR) sequence of the endogenous GOI encoding the endogenous POI and [3′-HR] is a 3′-nucleic acid sequence homology region comprising homology to the genomic locus immediately downstream (3′) of the endogenous WT-5′-UTR sequence of the endogenous GOI encoding the endogenous POI, wherein the 5′-HR and 3′-HR comprise sufficient homology to said genomic loci to effect integration of the introduced mod-5′-UTR polynucleotide construct into the genome of the modified Bacillus cell by homologous recombination, thereby replacing the endogenous WT-5′-UTR with the mod-5′-UTR of SEQ ID NO: 2, and
(c) cultivating the modified Bacillus sp. cell of step (b) in a medium suitable for the production of the endogenous POI,
wherein the modified cell of step (c) produces an increased amount of the endogenous POI relative to the parental cell of step (a) when cultivated under similar conditions.


SEQUENCE LISTING (21 sequences)

SEQ ID NO: 1 - DNA, 58 bp, Bacillus subtilis
acagaatagtcttttaagtaagtctactctgaatttttttaaaaggagagggtaaaga

SEQ ID NO: 2 - DNA, 57 bp, Artificial Sequence
  Modified 5′-UTR
acagaatagtcttttaagtaagtctactctgaatttttttaaaaggagagggtaaag

SEQ ID NO: 3 - DNA, 579 bp, Artificial Sequence
  comK
atgagcacagaggatatgacaaaggatacgtatgaagtaaacagttcgacaatggctgtcctgcctctgggtgagggggagaaatccgcctcaaaaatacttgagaccgacaggactttccgcgtcaatatgaagccgtttcaaattatcgaaagaagctgccgctatttcggatcgagctatgcgggaagaaaagcgggcacatatgaagtcattaaagtttcccataaaccgccgatcatggtggatcactcaaacaacatttttcttttccccacattttcctcaactcgtcctcagtgcgggtggctttcccatgcgcatgttcacgagttttgcgcggcaaagtatgacaacacgtttgtcacgtttgtcaacggggaaacgctggagctgcccgtatccatctcatctttcgaaaaccaggtttaccgaacggcatggctgagaacaaaatttatcgacaggattgaaggaaaccccatgcagaagaaacaggaatttatgctctatccgaaagaagaccggaatcagctgatatacgaattcatcctcagggagctgaaaaagcgctattga

SEQ ID NO: 4 - DNA, 6208 bp, Artificial Sequence
  synthetic WT-5′-UTR construct
ggctatgacgaaacgattgcagcctatgaggcgagtctcgaaaagctcggacttgactaccttgatttatacctgatccactggcctgttgaaggacgctacaaagcggcgtggaaagcgcttgaaacactttatgaacaaggacgcgtaaaagcaatcggagtgagcaattttcagattcaccatctggaagacttgctgaaagatgccgccgtcaaaccggcgatcaaccaggttgagtatcatccgcggctgacgcagaaagagctgcaagcgttttgccgtgcgcacggcatccagctgcaagcatggtcgccgctgatgcaaggccaattgctcagccatccactgctgaaagatatcgcggacaagtacggcaagacaccggcccaagtcattttgcgctgggatttgcaaaacggggtcgttacgattccgaagtcgactaaagcggagcggattgcccaaaacgcggacatatttgattttgaactgaccaccgaggaaatgaagcaaattgacgcgctgaatgaaaacacccgtgtcggccctgatcccgataactttgacttttaacaaaacggccccgttcgacattcgaacggggctttaattgaattgtgcggttacaccgccggactccatcatcatcagttcttttttcatatccaatccgccccggtatcccgtgagctgcccgcttttaccgataacccgatggcaaggcaccaccattaacagcggatttgcgccgatcgccgcgcctactgcccgcacagcggcctgcttttcaatatgctcggcgatatcggaataggagcaagtgctgccgtaagggatttcggagagcgccttccacactgccagctgaaaaggcgtgccggcaaggtcgacaggaaagctgaaatgagttcgcttgccgttcaaatacgcctgcagctgctcggcgtattctgccaatcctttgtcatcccgaatgaaaactggctgtgtaaatcttttttcagcccaagcggccaaatcctcgaagccttgattccatccccctgtaaaacagagcccgcgggcagtcgccccaatgtgaatctgccaacctcggcaaataagcgtacgccagtatacgatttgatcgtccatatgtttacctccgtttcatttgccggtacgacgtcggcgattgcccagtcttctttttaaacaaagaggcaaaatattccgcattcgcaatgcctaccattgaagcgatttctgcgatcgatcgttctgaatgagcaagcaaatcgaccgctttctcaatccttttctgcaggatgtattctgccggcgagacgcctttgattcgtttaaatgtccgctgcaggtgaaaagggctgatatggcacctgtcagccaaagcttgcagagacagcggatcgcgataagattcctcgatgatttccaccacacgctgtgccagctcttcatccggcagcagcgccccggccggattgcagcgtttgcaggggcggtacccttctgataaagcatcttttgcattgaaaaagatctgcacattgtcgatttgcggaactctcgatttgcaggaagggcggcaaaatatgccggtcgttttgaccgcgtaataaaaaactccgtcataggcggaatcgttttccgtaatcgcccgccacatttcaggcgtcaatcgtgatttgctgttcatatcttcaccccgatctatgtcagtataacctatatgacagccggaggtggagaggcggagaacggcacagcaagaagacaaagaagaagagagactgttgcctggacctccgaaacgcgctacaattcatttacaacacaggatggggtgagaatattgccggaatcagtgaagcaggcctcctaaaataaaaatctatattttaggaggtaaaacatgaattttcaaacaatcgagcttgacacatggtatagaaaatcttattttgaccattacatgaaggaagcgaaatgttctttcagcatcacggcaaacgtcaatgtgacaaatttgctcgccgtgctcaagaaaaagaagctcaagctgtatccggcttttatttatatcgtatcaagggtcattcattcgcgccctgagtttagaacaacgtttgatgacaaaggacagctgggttattgggaacaaatgcatccgtgctatgcgatttttcatcaggacgaccaaacgttttccgccctctggacggaatactcagacgatttttcgcagttttatcatcaatatcttctggacgccgagcgctttggagacaaaaggggcctttgggctaagccggacatcccgcccaatacgttttcagtttcttctattccatgggtgcgcttttcaaacttcaatttaaaccttgataacagcgaacacttgctgccgattattacaaacgggaaatacttttcagaaggcagggaaacatttttgcccgtttccttgcaagttcaccatgcagtgtgtgacggctatcatgccggcgcttttataaacgagttggaacggcttgccgccgattgtgaggagtggcttgtgtgacagaggaaaggccgatatgattcggccttttttatatgtacttcttagcgggtctcttaacccccctcgaggtcgctgataaacagctgacatcaatatcctattttttcaaaaaatattttaaaaagttgttgacttaaaagaagctaaatgttatagtaataaaacagaatagtcttttaagtaagtctactctgaatttttttaaaaggagagggtaaagaatgaaacaacaaaaacggctttacgcccgattgctgacgctgttatttgcgctcatcttcttgctgcctcattctgcagctagcgcagccgcaccgtttaacggtaccatgatgcagtattttgaatggtacttgccggatgatggcacgttatggaccaaagtggccaatgaagccaacaacttatccagccttggcatcaccgctctttggctgccgcccgcttacaaaggaacaagccgcagcgacgtagggtacggagtatacgacttgtatgacctcggcgaattcaatcaaaaagggaccgtccgcacaaaatatggaacaaaagctcaatatcttcaagccattcaagccgcccacgccgctggaatgcaagtgtacgccgatgtcgtgttcgaccataaaggcggcgctgacggcacggaatgggtggacgccgtcgaagtcaatccgtccgaccgcaaccaagaaatctcgggcacctatcaaatccaagcatggacgaaatttgattttcccgggcggggcaacacctactccagctttaagtggcgctggtaccattttgacggcgttgattgggacgaaagccgaaaattaagccgcatttacaaattcaggggcatcggcaaagcgtgggattggccggtagacacagaaaacggaaactatgactacttaatgtatgccgaccttgatatggatcatcccgaagtcgtgaccgagctgaaaaactgggggaaatggtatgtcaacacaacgaacattgatgggttccggcttgatgccgtcaagcatattaagttcagtttttttcctgattggttgtcgtatgtgcgttctcagactggcaagccgctatttaccgtcggggaatattggagctatgacatcaacaagttgcacaattacattacgaaaacaaacggaacgatgtctttgtttgatgccccgttacacaacaaattttataccgcttccaaatcagggggcgcatttgatatgcgcacgttaatgaccaatactctcatgaaagatcaaccgacattggccgtcaccttcgttgataatcatgacaccgaacccggccaagcgcttcagtcatgggtcgacccatggttcaaaccgttggcttacgcctttattctaactcggcaggaaggatacccgtgcgtcttttatggtgactattatggcattccacaatataacattccttcgctgaaaagcaaaatcgatccgctcctcatcgcgcgcagggattatgcttacggaacgcaacatgattatcttgatcactccgacatcatcgggtggacaagggaaggggtcactgaaaaaccaggatccgggctggccgcactgatcaccgatgggccgggaggaagcaaatggatgtacgttggcaaacaacacgctggaaaagtgttctatgaccttaccggcaaccggagtgacaccgtcaccatcaacagtgatggatggggggaattcaaagtcaatggcggttcggtttcggtttgggttcctagaaaaacgacctaaaagcttctcgaggttaacagaggacggatttcctgaaggaaatccgtttttttatttttaacatctctcactgctgtgtgattttactcacggcatttggaacgccggctctcaacaaactttctgtagtgaaaatcatgaaccaaacggatcgtcggcctgattaacagctgaaagctgccgatcacaaacatccatagtcccgccggcttcagttcctcggagaaaaagcagaagctcccgacaaggaataaaaggccgatgagaaaatcgtttaatgtatgtagaactttgtatctttttttgaaaaagagttcatatcgattgttattgttttgcggcattgcttgatcactccaatccttttatttaccctgccggaagccggagtgaaacgccggtatacataggatttatgaattaggaaaacatatggggaaataaaccatccaggagtgaaaaatatgcggttattcatatgtgcatcgtgcctgttcggcttgattgttccgtcatttgaaacgaaagcgctgacgtttgaagaattgccggttaaacaagcttcaaaacaatgggaagttcaaatcggtaaagccgaagccggaaacggaatggcgaaaccggaaaaaggagcgtttcatacttatgctgtcgaaatcaaaaacattggacacgatgtggcttcggcggaaatttttgtctatcggaacgagcctaattcttcaacgaaattttcgctttggaacattcctcacgaaaatccggtttctttagccaaaagcttaaatcacggaagctctgtcaagcaccgcaatctgcttatggcagagaatgcgaccgaattggaagtggacatgatttggacggaaaaaggaagcgaaggcagacttttaaaggaaacgttcattttcaagggagatgaatcatgaagaaaaaatggccgttcatcgtcaacggtctttttttaatgacttaggcagccgatcgttcggccatacgatatcgaagcgacctcgaaccagcagagctcgtcacaaaacatttgcatttaaagaaaaatacaggatgttttcaccaatatttttctcaatgatgatacactattgacaagctgctactttgggagggtgtttccatagatgccgatgaagcaaaaacaccaaatgtgtcatgagagctctctctaatcgatataaaagtagggtgaaccggggttgtcaatctgtaaaagatctttttttatcccgtgatacgcttttggaattctgaatcttcaagaaagtccccagccttttgctgatcaatcgagaacaaaggatgatacatatgaaaagaatagataaaatctaccatcagctgctggataattttcgcgaaaagaatatcaatcagcttttaaagatacaagggaattcggctaaagaaatcgccgggcagctgcaaatggagcgttccaatgtcagctttgaattaaacaatctggttcgggccaaaaaggtgatcaagattaaaacgttccccgtccgctacatcccggtggaaattgttgaaaacgtcttgaacatcaaatggaattcagagttgatggaggttgaagaactgaggcggctggctgacggccaaaaaaagccggcgcgcaatatatccgccgatcccctcgagctcatgatcggggctaaagggagcttgaaaaaggcaatttctcaggcgaaagcggcagtcttttatcctccgcacggcttgcatatgctgctgctcgggccgacgggttcggggaaatcgctgtttgcgaatcggatctaccagttcgccgtttattctgacatattgaagcccgattccccgttcatcacattcaactgtgcag

SEQ ID NO: 5 - DNA, 6207 bp, Artificial Sequence
  synthetic mod-5-UTR construct
ggctatgacgaaacgattgcagcctatgaggcgagtctcgaaaagctcggacttgactaccttgatttatacctgatccactggcctgttgaaggacgctacaaagcggcgtggaaagcgcttgaaacactttatgaacaaggacgcgtaaaagcaatcggagtgagcaattttcagattcaccatctggaagacttgctgaaagatgccgccgtcaaaccggcgatcaaccaggttgagtatcatccgcggctgacgcagaaagagctgcaagcgttttgccgtgcgcacggcatccagctgcaagcatggtcgccgctgatgcaaggccaattgctcagccatccactgctgaaagatatcgcggacaagtacggcaagacaccggcccaagtcattttgcgctgggatttgcaaaacggggtcgttacgattccgaagtcgactaaagcggagcggattgcccaaaacgcggacatatttgattttgaactgaccaccgaggaaatgaagcaaattgacgcgctgaatgaaaacacccgtgtcggccctgatcccgataactttgacttttaacaaaacggccccgttcgacattcgaacggggctttaattgaattgtgcggttacaccgccggactccatcatcatcagttcttttttcatatccaatccgccccggtatcccgtgagctgcccgcttttaccgataacccgatggcaaggcaccaccattaacagcggatttgcgccgatcgccgcgcctactgcccgcacagcggcctgcttttcaatatgctcggcgatatcggaataggagcaagtgctgccgtaagggatttcggagagcgccttccacactgccagctgaaaaggcgtgccggcaaggtcgacaggaaagctgaaatgagttcgcttgccgttcaaatacgcctgcagctgctcggcgtattctgccaatcctttgtcatcccgaatgaaaactggctgtgtaaatcttttttcagcccaagcggccaaatcctcgaagccttgattccatccccctgtaaaacagagcccgcgggcagtcgccccaatgtgaatctgccaacctcggcaaataagcgtacgccagtatacgatttgatcgtccatatgtttacctccgtttcatttgccggtacgacgtcggcgattgcccagtcttctttttaaacaaagaggcaaaatattccgcattcgcaatgcctaccattgaagcgatttctgcgatcgatcgttctgaatgagcaagcaaatcgaccgctttctcaatccttttctgcaggatgtattctgccggcgagacgcctttgattcgtttaaatgtccgctgcaggtgaaaagggctgatatggcacctgtcagccaaagcttgcagagacagcggatcgcgataagattcctcgatgatttccaccacacgctgtgccagctcttcatccggcagcagcgccccggccggattgcagcgtttgcaggggcggtacccttctgataaagcatcttttgcattgaaaaagatctgcacattgtcgatttgcggaactctcgatttgcaggaagggcggcaaaatatgccggtcgttttgaccgcgtaataaaaaactccgtcataggcggaatcgttttccgtaatcgcccgccacatttcaggcgtcaatcgtgatttgctgttcatatcttcaccccgatctatgtcagtataacctatatgacagccggaggtggagaggcggagaacggcacagcaagaagacaaagaagaagagagactgttgcctggacctccgaaacgcgctacaattcatttacaacacaggatggggtgagaatattgccggaatcagtgaagcaggcctcctaaaataaaaatctatattttaggaggtaaaacatgaattttcaaacaatcgagcttgacacatggtatagaaaatcttattttgaccattacatgaaggaagcgaaatgttctttcagcatcacggcaaacgtcaatgtgacaaatttgctcgccgtgctcaagaaaaagaagctcaagctgtatccggcttttatttatatcgtatcaagggtcattcattcgcgccctgagtttagaacaacgtttgatgacaaaggacagctgggttattgggaacaaatgcatccgtgctatgcgatttttcatcaggacgaccaaacgttttccgccctctggacggaatactcagacgatttttcgcagttttatcatcaatatcttctggacgccgagcgctttggagacaaaaggggcctttgggctaagccggacatcccgcccaatacgttttcagtttcttctattccatgggtgcgcttttcaaacttcaatttaaaccttgataacagcgaacacttgctgccgattattacaaacgggaaatacttttcagaaggcagggaaacatttttgcccgtttccttgcaagttcaccatgcagtgtgtgacggctatcatgccggcgcttttataaacgagttggaacggcttgccgccgattgtgaggagtggcttgtgtgacagaggaaaggccgatatgattcggccttttttatatgtacttcttagcgggtctcttaacccccctcgaggtcgctgataaacagctgacatcaatatcctattttttcaaaaaatattttaaaaagttgttgacttaaaagaagctaaatgttatagtaataaaacagaatagtcttttaagtaagtctactctgaatttttttaaaaggagagggtaaagatgaaacaacaaaaacggctttacgcccgattgctgacgctgttatttgcgctcatcttcttgctgcctcattctgcagctagcgcagccgcaccgtttaacggtaccatgatgcagtattttgaatggtacttgccggatgatggcacgttatggaccaaagtggccaatgaagccaacaacttatccagccttggcatcaccgctctttggctgccgcccgcttacaaaggaacaagccgcagcgacgtagggtacggagtatacgacttgtatgacctcggcgaattcaatcaaaaagggaccgtccgcacaaaatatggaacaaaagctcaatatcttcaagccattcaagccgcccacgccgctggaatgcaagtgtacgccgatgtcgtgttcgaccataaaggcggcgctgacggcacggaatgggtggacgccgtcgaagtcaatccgtccgaccgcaaccaagaaatctcgggcacctatcaaatccaagcatggacgaaatttgattttcccgggcggggcaacacctactccagctttaagtggcgctggtaccattttgacggcgttgattgggacgaaagccgaaaattaagccgcatttacaaattcaggggcatcggcaaagcgtgggattggccggtagacacagaaaacggaaactatgactacttaatgtatgccgaccttgatatggatcatcccgaagtcgtgaccgagctgaaaaactgggggaaatggtatgtcaacacaacgaacattgatgggttccggcttgatgccgtcaagcatattaagttcagtttttttcctgattggttgtcgtatgtgcgttctcagactggcaagccgctatttaccgtcggggaatattggagctatgacatcaacaagttgcacaattacattacgaaaacaaacggaacgatgtctttgtttgatgccccgttacacaacaaattttataccgcttccaaatcagggggcgcatttgatatgcgcacgttaatgaccaatactctcatgaaagatcaaccgacattggccgtcaccttcgttgataatcatgacaccgaacccggccaagcgcttcagtcatgggtcgacccatggttcaaaccgttggcttacgcctttattctaactcggcaggaaggatacccgtgcgtcttttatggtgactattatggcattccacaatataacattccttcgctgaaaagcaaaatcgatccgctcctcatcgcgcgcagggattatgcttacggaacgcaacatgattatcttgatcactccgacatcatcgggtggacaagggaaggggtcactgaaaaaccaggatccgggctggccgcactgatcaccgatgggccgggaggaagcaaatggatgtacgttggcaaacaacacgctggaaaagtgttctatgaccttaccggcaaccggagtgacaccgtcaccatcaacagtgatggatggggggaattcaaagtcaatggcggttcggtttcggtttgggttcctagaaaaacgacctaaaagcttctcgaggttaacagaggacggatttcctgaaggaaatccgtttttttatttttaacatctctcactgctgtgtgattttactcacggcatttggaacgccggctctcaacaaactttctgtagtgaaaatcatgaaccaaacggatcgtcggcctgattaacagctgaaagctgccgatcacaaacatccatagtcccgccggcttcagttcctcggagaaaaagcagaagctcccgacaaggaataaaaggccgatgagaaaatcgtttaatgtatgtagaactttgtatctttttttgaaaaagagttcatatcgattgttattgttttgcggcattgcttgatcactccaatccttttatttaccctgccggaagccggagtgaaacgccggtatacataggatttatgaattaggaaaacatatggggaaataaaccatccaggagtgaaaaatatgcggttattcatatgtgcatcgtgcctgttcggcttgattgttccgtcatttgaaacgaaagcgctgacgtttgaagaattgccggttaaacaagcttcaaaacaatgggaagttcaaatcggtaaagccgaagccggaaacggaatggcgaaaccggaaaaaggagcgtttcatacttatgctgtcgaaatcaaaaacattggacacgatgtggcttcggcggaaatttttgtctatcggaacgagcctaattcttcaacgaaattttcgctttggaacattcctcacgaaaatccggtttctttagccaaaagcttaaatcacggaagctctgtcaagcaccgcaatctgcttatggcagagaatgcgaccgaattggaagtggacatgatttggacggaaaaaggaagcgaaggcagacttttaaaggaaacgttcattttcaagggagatgaatcatgaagaaaaaatggccgttcatcgtcaacggtctttttttaatgacttaggcagccgatcgttcggccatacgatatcgaagcgacctcgaaccagcagagctcgtcacaaaacatttgcatttaaagaaaaatacaggatgttttcaccaatatttttctcaatgatgatacactattgacaagctgctactttgggagggtgtttccatagatgccgatgaagcaaaaacaccaaatgtgtcatgagagctctctctaatcgatataaaagtagggtgaaccggggttgtcaatctgtaaaagatctttttttatcccgtgatacgcttttggaattctgaatcttcaagaaagtccccagccttttgctgatcaatcgagaacaaaggatgatacatatgaaaagaatagataaaatctaccatcagctgctggataattttcgcgaaaagaatatcaatcagcttttaaagatacaagggaattcggctaaagaaatcgccgggcagctgcaaatggagcgttccaatgtcagctttgaattaaacaatctggttcgggccaaaaaggtgatcaagattaaaacgttccccgtccgctacatcccggtggaaattgttgaaaacgtcttgaacatcaaatggaattcagagttgatggaggttgaagaactgaggcggctggctgacggccaaaaaaagccggcgcgcaatatatccgccgatcccctcgagctcatgatcggggctaaagggagcttgaaaaaggcaatttctcaggcgaaagcggcagtcttttatcctccgcacggcttgcatatgctgctgctcgggccgacgggttcggggaaatcgctgtttgcgaatcggatctaccagttcgccgtttattctgacatattgaagcccgattccccgttcatcacattcaactgtgcag

SEQ ID NO: 6 - DNA, 1702 bp, Artificial Sequence
  5′-HR to catH locus
ggctatgacgaaacgattgcagcctatgaggcgagtctcgaaaagctcggacttgactaccttgatttatacctgatccactggcctgttgaaggacgctacaaagcggcgtggaaagcgcttgaaacactttatgaacaaggacgcgtaaaagcaatcggagtgagcaattttcagattcaccatctggaagacttgctgaaagatgccgccgtcaaaccggcgatcaaccaggttgagtatcatccgcggctgacgcagaaagagctgcaagcgttttgccgtgcgcacggcatccagctgcaagcatggtcgccgctgatgcaaggccaattgctcagccatccactgctgaaagatatcgcggacaagtacggcaagacaccggcccaagtcattttgcgctgggatttgcaaaacggggtcgttacgattccgaagtcgactaaagcggagcggattgcccaaaacgcggacatatttgattttgaactgaccaccgaggaaatgaagcaaattgacgcgctgaatgaaaacacccgtgtcggccctgatcccgataactttgacttttaacaaaacggccccgttcgacattcgaacggggctttaattgaattgtgcggttacaccgccggactccatcatcatcagttcttttttcatatccaatccgccccggtatcccgtgagctgcccgcttttaccgataacccgatggcaaggcaccaccattaacagcggatttgcgccgatcgccgcgcctactgcccgcacagcggcctgcttttcaatatgctcggcgatatcggaataggagcaagtgctgccgtaagggatttcggagagcgccttccacactgccagctgaaaaggcgtgccggcaaggtcgacaggaaagctgaaatgagttcgcttgccgttcaaatacgcctgcagctgctcggcgtattctgccaatcctttgtcatcccgaatgaaaactggctgtgtaaatcttttttcagcccaagcggccaaatcctcgaagccttgattccatccccctgtaaaacagagcccgcgggcagtcgccccaatgtgaatctgccaacctcggcaaataagcgtacgccagtatacgatttgatcgtccatatgtttacctccgtttcatttgccggtacgacgtcggcgattgcccagtcttctttttaaacaaagaggcaaaatattccgcattcgcaatgcctaccattgaagcgatttctgcgatcgatcgttctgaatgagcaagcaaatcgaccgctttctcaatccttttctgcaggatgtattctgccggcgagacgcctttgattcgtttaaatgtccgctgcaggtgaaaagggctgatatggcacctgtcagccaaagcttgcagagacagcggatcgcgataagattcctcgatgatttccaccacacgctgtgccagctcttcatccggcagcagcgccccggccggattgcagcgtttgcaggggcggtacccttctgataaagcatcttttgcattgaaaaagatctgcacattgtcgatttgcggaactctcgatttgcaggaagggcggcaaaatatgccggtcgttttgaccgcgtaataaaaaactccgtcataggcggaatcgttttccgtaatcgcccgccacatttcaggcgtcaatcgtgatttgctgttcat

SEQ ID NO: 7 - DNA, 938 bp, Artificial Sequence
  catH gene
atcttcaccccgatctatgtcagtataacctatatgacagccggaggtggagaggcggagaacggcacagcaagaagacaaagaagaagagagactgttgcctggacctccgaaacgcgctacaattcatttacaacacaggatggggtgagaatattgccggaatcagtgaagcaggcctcctaaaataaaaatctatattttaggaggtaaaacatgaattttcaaacaatcgagcttgacacatggtatagaaaatcttattttgaccattacatgaaggaagcgaaatgttctttcagcatcacggcaaacgtcaatgtgacaaatttgctcgccgtgctcaagaaaaagaagctcaagctgtatccggcttttatttatatcgtatcaagggtcattcattcgcgccctgagtttagaacaacgtttgatgacaaaggacagctgggttattgggaacaaatgcatccgtgctatgcgatttttcatcaggacgaccaaacgttttccgccctctggacggaatactcagacgatttttcgcagttttatcatcaatatcttctggacgccgagcgctttggagacaaaaggggcctttgggctaagccggacatcccgcccaatacgttttcagtttcttctattccatgggtgcgcttttcaaacttcaatttaaaccttgataacagcgaacacttgctgccgattattacaaacgggaaatacttttcagaaggcagggaaacatttttgcccgtttccttgcaagttcaccatgcagtgtgtgacggctatcatgccggcgcttttataaacgagttggaacggcttgccgccgattgtgaggagtggcttgtgtgacagaggaaaggccgatatgattcggccttttttatatgtacttcttagcgggtctcttaacccccctcgag

SEQ ID NO: 8 - DNA, 95 bp, Artificial Sequence
  spoVGrrnIp promoter
gtcgctgataaacagctgacatcaatatcctattttttcaaaaaatattttaaaaagttgttgacttaaaagaagctaaatgttatagtaataaa

SEQ ID NO: 9 - DNA, 87 bp, Artificial Sequence
  amylase signal sequence
atgaaacaacaaaaacggctttacgcccgattgctgacgctgttatttgcgctcatcttcttgctgcctcattctgcagctagcgca

SEQ ID NO: 10 - DNA, 1461 bp, Artificial Sequence
  variant amylase
gccgcaccgtttaacggtaccatgatgcagtattttgaatggtacttgccggatgatggcacgttatggaccaaagtggccaatgaagccaacaacttatccagccttggcatcaccgctctttggctgccgcccgcttacaaaggaacaagccgcagcgacgtagggtacggagtatacgacttgtatgacctcggcgaattcaatcaaaaagggaccgtccgcacaaaatatggaacaaaagctcaatatcttcaagccattcaagccgcccacgccgctggaatgcaagtgtacgccgatgtcgtgttcgaccataaaggcggcgctgacggcacggaatgggtggacgccgtcgaagtcaatccgtccgaccgcaaccaagaaatctcgggcacctatcaaatccaagcatggacgaaatttgattttcccgggcggggcaacacctactccagctttaagtggcgctggtaccattttgacggcgttgattgggacgaaagccgaaaattaagccgcatttacaaattcaggggcatcggcaaagcgtgggattggccggtagacacagaaaacggaaactatgactacttaatgtatgccgaccttgatatggatcatcccgaagtcgtgaccgagctgaaaaactgggggaaatggtatgtcaacacaacgaacattgatgggttccggcttgatgccgtcaagcatattaagttcagtttttttcctgattggttgtcgtatgtgcgttctcagactggcaagccgctatttaccgtcggggaatattggagctatgacatcaacaagttgcacaattacattacgaaaacaaacggaacgatgtctttgtttgatgccccgttacacaacaaattttataccgcttccaaatcagggggcgcatttgatatgcgcacgttaatgaccaatactctcatgaaagatcaaccgacattggccgtcaccttcgttgataatcatgacaccgaacccggccaagcgcttcagtcatgggtcgacccatggttcaaaccgttggcttacgcctttattctaactcggcaggaaggatacccgtgcgtcttttatggtgactattatggcattccacaatataacattccttcgctgaaaagcaaaatcgatccgctcctcatcgcgcgcagggattatgcttacggaacgcaacatgattatcttgatcactccgacatcatcgggtggacaagggaaggggtcactgaaaaaccaggatccgggctggccgcactgatcaccgatgggccgggaggaagcaaatggatgtacgttggcaaacaacacgctggaaaagtgttctatgaccttaccggcaaccggagtgacaccgtcaccatcaacagtgatggatggggggaattcaaagtcaatggcggttcggtttcggtttgggttcctagaaaaacgacctaa

SEQ ID NO: 11 - DNA, 58 bp, Artificial Sequence
  amylase terminator sequence
aagcttctcgaggttaacagaggacggatttcctgaaggaaatccgtttttttatttt

SEQ ID NO: 12 - DNA, 1809 bp, Artificial Sequence
  3′-HR to CatH locus
taacatctctcactgctgtgtgattttactcacggcatttggaacgccggctctcaacaaactttctgtagtgaaaatcatgaaccaaacggatcgtcggcctgattaacagctgaaagctgccgatcacaaacatccatagtcccgccggcttcagttcctcggagaaaaagcagaagctcccgacaaggaataaaaggccgatgagaaaatcgtttaatgtatgtagaactttgtatctttttttgaaaaagagttcatatcgattgttattgttttgcggcattgcttgatcactccaatccttttatttaccctgccggaagccggagtgaaacgccggtatacataggatttatgaattaggaaaacatatggggaaataaaccatccaggagtgaaaaatatgcggttattcatatgtgcatcgtgcctgttcggcttgattgttccgtcatttgaaacgaaagcgctgacgtttgaagaattgccggttaaacaagcttcaaaacaatgggaagttcaaatcggtaaagccgaagccggaaacggaatggcgaaaccggaaaaaggagcgtttcatacttatgctgtcgaaatcaaaaacattggacacgatgtggcttcggcggaaatttttgtctatcggaacgagcctaattcttcaacgaaattttcgctttggaacattcctcacgaaaatccggtttctttagccaaaagcttaaatcacggaagctctgtcaagcaccgcaatctgcttatggcagagaatgcgaccgaattggaagtggacatgatttggacggaaaaaggaagcgaaggcagacttttaaaggaaacgttcattttcaagggagatgaatcatgaagaaaaaatggccgttcatcgtcaacggtctttttttaatgacttaggcagccgatcgttcggccatacgatatcgaagcgacctcgaaccagcagagctcgtcacaaaacatttgcatttaaagaaaaatacaggatgttttcaccaatatttttctcaatgatgatacactattgacaagctgctactttgggagggtgtttccatagatgccgatgaagcaaaaacaccaaatgtgtcatgagagctctctctaatcgatataaaagtagggtgaaccggggttgtcaatctgtaaaagatctttttttatcccgtgatacgcttttggaattctgaatcttcaagaaagtccccagccttttgctgatcaatcgagaacaaaggatgatacatatgaaaagaatagataaaatctaccatcagctgctggataattttcgcgaaaagaatatcaatcagcttttaaagatacaagggaattcggctaaagaaatcgccgggcagctgcaaatggagcgttccaatgtcagctttgaattaaacaatctggttcgggccaaaaaggtgatcaagattaaaacgttccccgtccgctacatcccggtggaaattgttgaaaacgtcttgaacatcaaatggaattcagagttgatggaggttgaagaactgaggcggctggctgacggccaaaaaaagccggcgcgcaatatatccgccgatcccctcgagctcatgatcggggctaaagggagcttgaaaaaggcaatttctcaggcgaaagcggcagtcttttatcctccgcacggcttgcatatgctgctgctcgggccgacgggttcggggaaatcgctgtttgcgaatcggatctaccagttcgccgtttattctgacatattgaagcccgattccccgttcatcacattcaactgtgcag

SEQ ID NO: 13 - protein, 486 aa, Artificial Sequence
  variant amylase
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWPVDTENGNYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTT

SEQ ID NO: 14 - DNA, 1967 bp, Artificial Sequence
  colony pcr construct
tgtgtgacggctatcatgccggcgcttttataaacgagttggaacggcttgccgccgattgtgaggagtggcttgtgtgacagaggaaaggccgatatgattcggccttttttatatgtacttcttagcgggtctcttaacccccctcgaggtcgctgataaacagctgacatcaatatcctattttttcaaaaaatattttaaaaagttgttgacttaaaagaagctaaatgttatagtaataaaacagaatagtcttttaagtaagtctactctgaatttttttaaaaggagagggtaaagaatgaaacaacaaaaacggctttacgcccgattgctgacgctgttatttgcgctcatcttcttgctgcctcattctgcagctagcgcagccgcaccgtttaacggtaccatgatgcagtattttgaatggtacttgccggatgatggcacgttatggaccaaagtggccaatgaagccaacaacttatccagccttggcatcaccgctctttggctgccgcccgcttacaaaggaacaagccgcagcgacgtagggtacggagtatacgacttgtatgacctcggcgaattcaatcaaaaagggaccgtccgcacaaaatatggaacaaaagctcaatatcttcaagccattcaagccgcccacgccgctggaatgcaagtgtacgccgatgtcgtgttcgaccataaaggcggcgctgacggcacggaatgggtggacgccgtcgaagtcaatccgtccgaccgcaaccaagaaatctcgggcacctatcaaatccaagcatggacgaaatttgattttcccgggcggggcaacacctactccagctttaagtggcgctggtaccattttgacggcgttgattgggacgaaagccgaaaattaagccgcatttacaaattcaggggcatcggcaaagcgtgggattggccggtagacacagaaaacggaaactatgactacttaatgtatgccgaccttgatatggatcatcccgaagtcgtgaccgagctgaaaaactgggggaaatggtatgtcaacacaacgaacattgatgggttccggcttgatgccgtcaagcatattaagttcagtttttttcctgattggttgtcgtatgtgcgttctcagactggcaagccgctatttaccgtcggggaatattggagctatgacatcaacaagttgcacaattacattacgaaaacaaacggaacgatgtctttgtttgatgccccgttacacaacaaattttataccgcttccaaatcagggggcgcatttgatatgcgcacgttaatgaccaatactctcatgaaagatcaaccgacattggccgtcaccttcgttgataatcatgacaccgaacccggccaagcgcttcagtcatgggtcgacccatggttcaaaccgttggcttacgcctttattctaactcggcaggaaggatacccgtgcgtcttttatggtgactattatggcattccacaatataacattccttcgctgaaaagcaaaatcgatccgctcctcatcgcgcgcagggattatgcttacggaacgcaacatgattatcttgatcactccgacatcatcgggtggacaagggaaggggtcactgaaaaaccaggatccgggctggccgcactgatcaccgatgggccgggaggaagcaaatggatgtacgttggcaaacaacacgctggaaaagtgttctatgaccttaccggcaaccggagtgacaccgtcaccatcaacagtgatggatggggggaattcaaagtcaatggcggttcggtttcggtttgggttcctagaaaaacgacctaaaagcttctcgaggttaacagaggacggatttcctgaaggaaatccgtttttttatttttaacatctctcactgctgtgtgattttactcacggcatttggaacgccggctctcaa

SEQ ID NO: 15 - DNA, 1966 bp, Artificial Sequence
  colony pcr construct -1A
tgtgtgacggctatcatgccggcgcttttataaacgagttggaacggcttgccgccgattgtgaggagtggcttgtgtgacagaggaaaggccgatatgattcggccttttttatatgtacttcttagcgggtctcttaacccccctcgaggtcgctgataaacagctgacatcaatatcctattttttcaaaaaatattttaaaaagttgttgacttaaaagaagctaaatgttatagtaataaaacagaatagtcttttaagtaagtctactctgaatttttttaaaaggagagggtaaagatgaaacaacaaaaacggctttacgcccgattgctgacgctgttatttgcgctcatcttcttgctgcctcattctgcagctagcgcagccgcaccgtttaacggtaccatgatgcagtattttgaatggtacttgccggatgatggcacgttatggaccaaagtggccaatgaagccaacaacttatccagccttggcatcaccgctctttggctgccgcccgcttacaaaggaacaagccgcagcgacgtagggtacggagtatacgacttgtatgacctcggcgaattcaatcaaaaagggaccgtccgcacaaaatatggaacaaaagctcaatatcttcaagccattcaagccgcccacgccgctggaatgcaagtgtacgccgatgtcgtgttcgaccataaaggcggcgctgacggcacggaatgggtggacgccgtcgaagtcaatccgtccgaccgcaaccaagaaatctcgggcacctatcaaatccaagcatggacgaaatttgattttcccgggcggggcaacacctactccagctttaagtggcgctggtaccattttgacggcgttgattgggacgaaagccgaaaattaagccgcatttacaaattcaggggcatcggcaaagcgtgggattggccggtagacacagaaaacggaaactatgactacttaatgtatgccgaccttgatatggatcatcccgaagtcgtgaccgagctgaaaaactgggggaaatggtatgtcaacacaacgaacattgatgggttccggcttgatgccgtcaagcatattaagttcagtttttttcctgattggttgtcgtatgtgcgttctcagactggcaagccgctatttaccgtcggggaatattggagctatgacatcaacaagttgcacaattacattacgaaaacaaacggaacgatgtctttgtttgatgccccgttacacaacaaattttataccgcttccaaatcagggggcgcatttgatatgcgcacgttaatgaccaatactctcatgaaagatcaaccgacattggccgtcaccttcgttgataatcatgacaccgaacccggccaagcgcttcagtcatgggtcgacccatggttcaaaccgttggcttacgcctttattctaactcggcaggaaggatacccgtgcgtcttttatggtgactattatggcattccacaatataacattccttcgctgaaaagcaaaatcgatccgctcctcatcgcgcgcagggattatgcttacggaacgcaacatgattatcttgatcactccgacatcatcgggtggacaagggaaggggtcactgaaaaaccaggatccgggctggccgcactgatcaccgatgggccgggaggaagcaaatggatgtacgttggcaaacaacacgctggaaaagtgttctatgaccttaccggcaaccggagtgacaccgtcaccatcaacagtgatggatggggggaattcaaagtcaatggcggttcggtttcggtttgggttcctagaaaaacgacctaaaagcttctcgaggttaacagaggacggatttcctgaaggaaatccgtttttttatttttaacatctctcactgctgtgtgattttactcacggcatttggaacgccggctctcaa

SEQ ID NO: 16 - DNA, 20 bp, Artificial Sequence
  primer
tgtgtgacggctatcatgcc

SEQ ID NO: 17 - DNA, 17 bp, Artificial Sequence
  primer
ttgagagccggcgttcc

SEQ ID NO: 18 - DNA, 20 bp, Artificial Sequence
  primer
aacgagttggaacggcttgc

SEQ ID NO: 19 - DNA, 20 bp, Artificial Sequence
  primer
ggcaacacctactccagctt

SEQ ID NO: 20 - DNA, 20 bp, Artificial Sequence
  primer
gatcactccgacatcatcgg

SEQ ID NO: 21 - protein, 192 aa, Artificial Sequence
  comK protein
MSTEDMTKDTYEVNSSTMAVLPLGEGEKSASKILETDRTFRVNMKPFQIIERSCRYFGSSYAGRKAGTYEVIKVSHKPPIMVDHSNNIFLFPTFSSTRPQCGWLSHAHVHEFCAAKYDNTFVTFVNGETLELPVSISSFENQVYRTAWLRTKFIDRIEGNPMQKKQEFMLYPKEDRNQLIYEFILRELKKRY